(19) United States
(12) Patent Application Publication
(10) Pub. No.: US 2023/0240849 A1
(43) Pub. Date: Aug. 3, 2023

(54) VALVE DELIVERY SYSTEM

(71) Applicant: SHIFAMED HOLDINGS, LLC, Campbell, CA (US)

(72) Inventors: Andrew BACKUS, Santa Cruz, CA (US); Jasper Ellington ADAMEK-BOWERS, San Francisco, CA (US); Jordan SKARO, San Jose, CA (US); Jonathan OAKDEN, San Jose, CA (US)

(21) Appl. No.: 18/004,609

(22) PCT Filed: Jul. 7, 2021
(86) PCT No.: PCT/US2021/040623

§ 371 Date: Jan. 6, 2023

Related U.S. Application Data
(60) Provisional application No. 63/048,963, filed on Jul. 7, 2020.

(51) Int. Cl. A61F 2/24

(52) U.S. Cl. CPC A61F 2/2457; A61F 2/2466; A61B 17/0469

(57) ABSTRACT

Delivery systems and methods for delivering a valve anchor and a valve prosthesis to a native valve annulus are provided. The anchor can be deployed near the native valve annulus with a tether attached thereto. A portion of the tether can be positioned inferior to the deployed anchor in an inverted configuration for advantageous positioning for adjusting the anchor position and/or deployment of the valve prosthesis. A positioning tool can be tracked over the tether and used to properly position the anchor. The positioning tool can be configured to transition to a stiffened state that includes one or more bends configured to allow efficient positioning of the deployed anchor and to provide room for deploying the valve prosthesis. Once the anchor is properly positioned, the valve prosthesis can be deployed within the native valve annulus and within the valve anchor.

### CROSS REFERENCE TO RELATED APPLICATIONS

This application claims priority to U.S. Provisional Application No. 63/048,963, filed on Jul. 7, 2020, and entitled “VALVE DELIVERY SYSTEM,” the entirety of which is incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Blood flow between heart chambers is regulated by native valves—the mitral valve, the aortic valve, the pulmonary valve, and the tricuspid valve. Each of these valves are passive one-way valves which open and close in response to differential pressures. Patients with valvular disease have abnormal anatomy and/or function of at least one valve. For example, a valve may suffer from insufficiency, also referred to as regurgitation, when the valve does not fully close and allows blood to flow retrograde. Valve stenosis can cause a valve to fail to open properly. Other diseases may also lead to dysfunction of the valves. While medications may be used to treat the disease, in many cases the defective valve may need to be repaired or replaced at some point during the patient's lifetime. Existing valves and surgical repair and/or replacement procedures may have relatively high risks, limited lifespans, and/or be highly invasive. Some less-invasive transcatheter options are available, however these generally are limited to aortic valve procedures, are limited in patient-to-patient flexibility, and often take longer than is desirable to implant. It would therefore be desirable to provide a less invasive procedure for repair and replacement of heart valves, including the mitral valve, quicker surgical methods, and/or prosthetic valves that can accommodate a variety of individual patients.

Additionally, existing valve repair/replacement procedures are often complicated and time-consuming. Presently-available procedures often require the placement of more than one component—for example, a prosthetic valve and a mechanism to anchor it to the native anatomy. Such procedures generally utilize multiple delivery catheters to carry the various components and delivery of each component separately to the valve, which can be time-consuming (particularly if components are delivered sequentially), complicated, and/or dangerous. For example, some devices provide rotational anchoring elements to capture the native anatomy such as the chordae tendineae in order to reduce delivery time. However, such anchoring elements, often by design, capture and pull the chordae along during their rotation, which can torque or otherwise stress and damage the chordae during deployment of the anchor elements, resulting in the need for additional medical interventions for the patient. Moreover, such anchoring elements may require extrusion from a low-profile (e.g., elongated) delivery configuration to an expanded configuration at or near the native valve. In at least some instances, extrusion of the anchoring elements can be complicated and may not reliably deploy into the correct expanded configuration relative to the delivery device and/or the native anatomy. Incorrect deployment may result in additional time to retract and re-deploy the anchoring element, more complicated anchoring procedures, and/or damage to the native tissue. It would therefore be desirable to provide quicker, less-complicated, less dangerous, and more reliably deployable valve assemblies for valvular replacement and repair.

### SUMMARY

Described herein are delivery systems and methods for delivering a valve anchor and a valve prosthesis to a native valve annulus. The anchor can have a spiral shape and be deployed around the chordae and/or leaflets of the native valve annulus. A tether connected to the anchor can extend to outside of the heart and/or the patient's body. A valve delivery device can be tracked over the tether to expand the valve prosthesis into the native valve annulus and within the valve anchor. Opposing forces between the prosthetic valve and the anchor can secure the prosthetic valve in place within the annulus of the native valve.

Prior to deployment of the prosthetic valve, a positioning tool can be used to properly adjust a position of the deployed valve anchor toward a selected position. For example, it can be preferable for the valve anchor to be in axial alignment with a midsection of the valve prosthesis when the valve prosthesis is expanded and deployed. Further, it may be desirable for the valve anchor to be as close to a plane of the native valve annulus as possible, which may require raising the valve anchor from its originally deployed position. At least a portion of the tether can be positioned inferior to the deployed anchor prior to tracking of the positioning tool so that the positioning tool can be advantageously positioned for adjusting the anchor position. The tether can be positioned in an inverted configuration at least partially within the ventricle. The positioning tool can be configured to transition to a stiffened state that includes one or more bends that allow the positioning tool to efficiently translate force to the deployed anchor from a sub-annular position.

According to some aspects, a method for treating a diseased native valve in a patient includes: encircling chordae of the diseased native valve with an anchor, the anchor having a tether attached thereto; translating a portion of the tether through an annulus of the diseased native valve from a first chamber of the heart to a second chamber of the heart while the anchor is positioned around the chordae, wherein translating the tether causes the tether to form a bend within the second chamber; tracking a valve delivery device over the tether; and releasing a valve prosthesis from the valve delivery device into the annulus of the diseased native valve and within the anchor.

In these aspects, the method can further include delivering the anchor to the diseased native valve with an anchor delivery device, the anchor delivery device including a steerable catheter. In these aspects, translating the portion of the tether through the annulus of the diseased native valve can include: advancing the steerable catheter towards a plane of the anchor;

advancing the tether while maintaining its attachment to the anchor, generating slack in the tether that at least partially coils within the first chamber; and advancing the steerable catheter across the plane of the anchor to position at least a portion of the tether into the second chamber. In these aspects, advancing the steerable catheter can include advancing the steerable catheter to a position that is proximal to an apex of the second chamber. In these aspects, the method can further include retracting the anchor delivery device from the diseased native valve. In these aspects, the anchor delivery device can further include an anchor guide that is configured to translate within the steerable catheter, wherein the anchor guide includes an inner lumen for housing the anchor. In these aspects, the anchor guide can take on a curved shape during deployment of the anchor from the anchor guide. In these aspects, the tether can take on substantially a U-shaped bend within the second chamber when in a bent configuration. In these aspects, positioning the tether in the bent configuration can include translating the tether distally with respect to a steerable catheter to provide increased tether length within the second chamber. In these aspects, tracking the valve delivery device over the tether can include advancing a valve delivery catheter through the annulus of the diseased native valve. In these aspects, tracking the valve delivery device over the tether can include deploying a positioning tool over the bent tether. In these aspects, the method can further include distally advancing the positioning tool until a distal end of the positioning tool interfaces with an attachment that is attached to a proximal end of the anchor. In these aspects, the method can further include applying a compression force along the positioning tool to cause the positioning tool to stiffen. In these aspects, applying the compression force can include pulling the tether proximally to place tension on the tether. In these aspects, pulling the tether proximally to place tension on the tether can include using a handle to apply a controlled degree of tension on the tether. In these aspects, applying the compression force can cause the positioning tool to take on substantially a U-shaped bend sub-annular to the anchor. In these aspects, the method can further include releasably attaching a distal end of the positioning tool to the anchor. In these aspects, the method can further include adjusting a position of the anchor relative to the diseased native valve using the positioning tool. In these aspects, adjusting the position of the anchor can include pulling the positioning tool proximally to move the anchor toward the annulus of the diseased valve. In these aspects, the position of the anchor can be adjusted closer to the annulus of the diseased native valve. In these aspects, the position of the anchor can be adjusted so that the anchor sits in a plane that is perpendicular to a plane of a distal end of the valve delivery device. In these aspects, causing the tether to bend within the second chamber can include causing the tether to invert within the second chamber. In these aspects, the method can further include delivering the anchor to the diseased native valve with an anchor delivery system, the anchor delivery system including a steerable catheter.

According to some aspects, a delivery system for delivering a valve prosthesis to a diseased valve of a heart includes: a tether configured to connect to a valve anchor, the tether further configured to extend from a position outside of the heart at least through a first chamber of the heart and into a second chamber the heart; and a valve delivery catheter configured to extend over the tether and into the second chamber, the valve delivery catheter configured to hold the valve prosthesis therein and to release the valve prosthesis within the valve anchor while the tether is connected to the valve anchor.

In these aspects, the tether can be configured to take on substantially a U-shape within the second chamber of the heart. In these aspects, the tether can be releasably attached to the valve anchor. In these aspects, a distal end of the tether can be configured to be releasably attached to a proximal end of the valve anchor. In these aspects, the valve anchor can have a spiral shape, wherein the valve delivery catheter is configured to extend through a central opening of the valve anchor to align the valve prosthesis before release of the valve prosthesis. In these aspects, the valve delivery catheter can be configured to axially align a midsection of the valve prosthesis with the diseased valve. In these aspects, a distal end of the valve delivery catheter can include a nosecone having a port sized and shape to pass the tether therethrough, the port having a central axis that is concentric with a central axis of the valve delivery catheter. In these aspects, the system can further include a positioning tool configured to pass through the valve delivery catheter and extend over the tether, the positioning tool configured to connect to the valve anchor and control a position of the valve anchor after the valve anchor is deployed within the heart. In these aspects, the positioning tool can include one or more regions that are configured to bend to a pre-determined shape. In these aspects, the regions can have a relatively reduced bending stiffness. In these aspects, the one or more regions can include one or more cutouts configured to allow the regions to bend to the pre-determined shape when a compression force is applied to the positioning tool. In these aspects, the one or more regions can be configured to bend to the pre-determined shape upon tensioning of the tether therein. In these aspects, a first region can be configured to transition from a straight shape to a U-shape. In these aspects, a second region can be configured to transition from a straight to a curved shape that bends radially inward toward a center of the valve anchor. In these aspects, the positioning tool can include a distal edge that is configured to engage with a proximal edge of the valve anchor, or a proximal edge of an attachment attached to a proximal end of the valve anchor. In these aspects, the distal edge of the positioning tool can be beveled and be configured to engage with a correspondingly beveled edge of the proximal edge of the valve anchor, or the proximal edge of an attachment attached to a proximal end of the valve anchor. In these aspects, the system can further include an anchor delivery device that is configured to deploy the valve anchor within the heart prior to delivery of the valve prosthesis by the valve delivery catheter. In these aspects, the anchor delivery catheter can include a steerable catheter having a distal end that is configured to bend for positioning the valve anchor within the heart. In these aspects, the anchor delivery catheter can be configured to extend over the tether. In these aspects, the anchor delivery device can include a steerable catheter that is configured to position a portion of the tether from the first chamber of the heart into the second chamber the heart. In these aspects, the steerable catheter can be configured to place the tether in an inverted configuration within the second chamber of the heart. In these aspects, the steerable catheter can be configured to form a U-shaped portion of the tether within the second chamber of the heart. In these aspects, the anchor delivery catheter can be configured to extend through a central opening of the valve anchor. In these aspects, the valve delivery catheter can include an inner shaft and an outer sheath, wherein the valve prosthesis is compressed between the inner shaft and outer sheath. In these aspects, the inner shaft can be configured to accommodate a positioning tool that is configured to adjust a position of the valve anchor once the valve anchor is wrapped around the chordae of the diseased valve.

In these aspects, the positioning tool can be configured to translate within the inner shaft and extend from a distal end of the valve delivery catheter. In these aspects, proximal retraction of the valve delivery catheter can be configured to be proximally retracted to causes the valve prosthesis to expand.

According to some aspects, a delivery system for delivering a valve prosthesis to a diseased valve of the heart includes: a tether configured to be connected to a valve anchor that surrounds at least a portion of the chordae of the diseased valve, the tether further configured to extend from a position outside of the heart at least through a first chamber of the heart and into a second chamber the heart; and a positioning tool configured to adjust a position of the valve anchor that surrounds the at least a portion of the chordae, the positioning tool including an elongate body configured to track over the tether and interface with a proximal portion of the valve anchor, the positioning tool including one or more regions configured to bend and place the positioning tool in a predetermined shape when an axial compression force is applied to the positioning tool.

In these aspects, the predetermined shape can include an inversion that is configured to be sub-annularly positioned with respect to the valve anchor withing the second chamber of the heart. In these aspects, the predetermined shape can include substantially a U-shaped bend. In these aspects, the one or more regions can include one or more cutouts along a portion of a perimeter of the elongate body, wherein compression of the positioning tool reduces a gap width of the one or more cutouts. In these aspects, the predetermined shape of the positioning tool can include a second bend at a distal portion of the positioning tool that is bent radially inward toward a center of the valve anchor. In these aspects, the system can further include a valve delivery catheter configured to accommodate the positioning tool therein, wherein the positioning tool is configured to translate within and extend from a distal end of the valve delivery catheter. In these aspects, the valve delivery catheter can further accommodate the valve prosthesis therein. In these aspects, the valve delivery catheter can include an inner shaft within a central opening of the valve prosthesis, the inner shaft configured to accommodate the positioning tool therein. In these aspects, proximal retraction of the valve delivery catheter can cause the valve prosthesis to expand. In these aspects, the predetermined shape can be configured to transmit an applied force to the valve anchor in a direction toward a plane of the diseased valve to move the valve anchor toward the plane of the diseased valve. In these aspects, the positioning tool can be configured to bend and stiffen upon proximal pulling of the tether. In these aspects, the positioning tool can be configured to bend and stiffen upon distal pushing of the positioning tool.

According to some aspects, a method for treating a diseased native valve of a heart includes: deploying an anchor from an anchor delivery catheter such that the anchor encircles chordae of the diseased native valve, wherein, after the anchor is deployed, a tether extends from a distal end of the anchor delivery catheter and is attached to the anchor; translating the distal end of the anchor delivery catheter from a first chamber of the heart to a second chamber of the heart, wherein the distal end of the anchor delivery catheter is translated through a central opening of the deployed anchor; advancing the tether through the anchor delivery catheter until a loop of the tether is placed within the second chamber of the heart; and retracting the tether within the anchor delivery catheter until slack is removed from the tether, wherein removing the slack releases tension on the tether and causes the tether to assume an inverted configuration within the second chamber of the heart.

In these aspects, the method can further include retracting the anchor delivery catheter from the heart. In these aspects, the method can further include tracking a valve delivery catheter over the tether, the valve delivery catheter having a valve prosthesis stored therein. In these aspects, tracking the valve delivery catheter over the tether can include deploying a positioning tool over a portion of the tether within the second chamber of the heart. In these aspects, the positioning tool can be advanced until a distal end of the positioning tool engages with an attachment that is attached to a proximal end of the anchor. In these aspects, the method can further include adjusting a position of the deployed anchor by translating the positioning tool with the anchor engaged therewith. In these aspects, adjusting the position of the deployed anchor can include moving the anchor closer to annulus plane of the diseased valve. In these aspects, the method can further include releasing the valve prosthesis from the valve delivery catheter into an annulus of the diseased native valve and within the central opening of the anchor. In these aspects, the tether can include a U-shaped bend within the second chamber of the heart when the tether is in the inverted configuration. In these aspects, deploying the anchor from the anchor delivery catheter can include deploying the anchor from a distal end of the anchor guide, the anchor guide positioned within the anchor delivery catheter. In these aspects, the method can further include translating the anchor guide distally with respect to the anchor delivery catheter. In these aspects, the method can further include causing the anchor guide to take on a curved shape configured to facilitate deployment of the anchor around the chordae. In these aspects, the method can further include bending the anchor delivery catheter to steer the distal end of the anchor delivery catheter through the central opening of the anchor.

According to some aspects, a delivery system for delivering a valve prosthesis to a diseased valve includes: a delivery catheter including: an outer sheath; and a hollow inner shaft that defines a tether lumen extending therethrough, the tether lumen configured to receive a tether; and a nosecone includes a port at a distal end thereof that is axially aligned with the tether lumen and configured to receive the tether, the nosecone formed to reversibly couple with and extend from a distal portion of the delivery catheter, and to retain the valve prosthesis within the delivery catheter.

In these aspects, the tether lumen can be positioned concentric with the outer sheath. In these aspects, the port can be positioned concentric with the outer sheath, when the nosecone is coupled with the delivery catheter. In these aspects, the delivery system can further include an elongate positioning tool that is configured: to be tracked within the tether lumen and through the port over the tether to a distal portion thereof, and to adjust an orientation of a valve anchor relative to anatomy of a patient. In these aspects, the elongate positioning tool can include at least two regions that are preferentially bendable for adjustment of the orientation of the valve anchor. In these aspects, adjustment of the orientation can include formation of a first predetermined bend and a second predetermined bend in a first region and a second region of the at least two regions. In these aspects, the first predetermined bend or the second predetermined bend can include an angle from about 120 degrees to about 310 degrees. In these aspects, the first predetermined bend or the second predetermined bend can include an angle from about 70 degrees to about 100 degrees. In these aspects, the first predetermined bend or the second predetermined bend can include a radius of curvature from about 2 millimeters (mm) to about 20 mm. In these aspects, the at least two regions can have a relatively reduced compressive stiffness in relation to a remainder of the positioning tool. In these aspects, the at least two regions can be formed to bend upon application of compressive force to the positioning tool, along a longitudinal axis. In these aspects, a first region of the at least two regions can be spaced apart from a second region along a longitudinal axis of the positioning tool. In these aspects, a first region of the at least two regions can be spaced apart from a second region along an azimuthal axis of the positioning tool. In these aspects, the at least two regions can be located distal to the distal end of the delivery catheter, when the positioning tool is extended to the distal portion of the tether. In these aspects, the at least two regions can include a plurality of cutouts in an outer wall of the positioning tool. In these aspects, a distal end of the positioning tool can be shaped and sized to interact with the distal portion of the tether. In these aspects, the distal end of the positioning tool can be shaped and sized to interactive with a proximal end of the distal portion of the tether. In these aspects, the distal end of the positioning tool can include a bevel that is shaped and sized to interact with a corresponding bevel on a proximal end of the distal portion of the tether. In these aspects, the delivery system can further include a valve delivery member that: is shaped and sized (a) for placement within the distal portion of the delivery catheter and for relative movement therebetween, and (b) to carry the valve prosthesis, and has an inner shaft defining a valve delivery member lumen that is configured to receive the tether. In these aspects, the valve delivery member can include the nosecone, and wherein the port is a distal end of the valve delivery member lumen.

According to some aspects, a delivery system for delivering a valve prosthesis to a diseased valve includes: an outer shaft; a valve delivery member at a distal end of the outer sheath; and a hollow inner shaft positioned within the outer shaft and the valve delivery member, the hollow inner shaft concentric with the outer shaft and configured to pass a tether therethrough.

According to some aspects, a delivery system for delivering a valve prosthesis to a diseased valve includes: a delivery catheter including: an outer sheath that defines a valve lumen sized to carry the valve prosthesis in a collapsed state, and an inner shaft extending through the outer sheath and along a central axis of the outer sheath, the inner shaft defining a tether lumen configured to receive a tether; and a nosecone including a port at a distal end thereof that is concentric with the tether lumen, the nosecone couplable to a distal end of the delivery catheter for retaining the valve prosthesis within the delivery catheter, wherein the delivery catheter and the nosecone are decouplable for deployment of the valve prosthesis, the deployment including translation of the tether lumen and/or the port with respect to the tether.

In these aspects, the tether lumen can be positioned along a central axis of the delivery catheter. In these aspects, the inner shaft can be concentric with the outer sheath. In these aspects, the port can be centrally disposed at the distal end of the nosecone. In these aspects, the tether lumen and/or the port can be configured for translation with respect to the tether, while the tether is maintained in a substantially fixed position. In these aspects, the delivery system can further include a positioning tool having an elongate body with a proximal end for extension to a proximal portion of the delivery catheter, and a distal end for extension to a distal portion of the tether, wherein the positioning tool is configured to translate within the tether lumen and through the port along the tether, and to couple with the distal portion of the tether to maintain it in the substantially fixed position. In these aspects, the tether lumen can be configured for proximal translation with respect to the tether for the deployment of the valve prosthesis. In these aspects, the port can be configured for distal translation with respect to the tether for the deployment of the valve prosthesis. In these aspects, the delivery system can further include a valve delivery member that: includes an elongate body having an outer wall shaped and sized (a) for placement within a distal portion of the delivery catheter and for relative movement therebetween, and (b) to carry the valve prosthesis, and has an inner shaft concentric with the outer wall and defining a valve delivery lumen extending therethrough, the valve delivery lumen configured to receive the tether. In these aspects, the valve delivery member can include the nosecone, and wherein the port forms a distal end of the valve delivery member lumen. In these aspects, the valve prosthesis can be configured to for expansion to an expanded state during the deployment thereof.

According to some aspects, a method for treating a diseased native valve in a patient includes: tracking a delivery device over a tether into a first chamber of a heart, the tether coupled with an anchor near a native valve annulus of the heart; further tracking the delivery device over the tether into a second chamber of the heart to position a valve capsule carried by the delivery device across the native valve annulus; and exposing the valve capsule to deploy a valve prosthesis.

In these aspects, the method can further include advancing the tether into the second chamber while maintaining the coupling with the anchor. In these aspects, advancing the tether can include forming a first bend and a second bend in the tether within the second chamber. In these aspects, one of the first bend or the second bend can include an angle from about 120 degrees to about 310 degrees. In these aspects, advancing the tether can be between the tracking the delivery device into the first chamber and the tracking the delivery device into the second chamber. In these aspects, advancing the tether can include advancing through an inner diameter of the anchor. In these aspects, advancing can be such that a majority of the tether extending from the delivery device is sub-annular. In these aspects, the anchor can be initially in a first position, further including moving the anchor to a second position. In these aspects, the moving the anchor can be between the tracking the delivery device into the second chamber and the exposing the valve capsule. In these aspects, the method can further include tracking a positioning tool over the tether such that a distal end of the positioning tool is positioned near the coupling of the tether and the anchor. In these aspects, the distal end of the positioning tool can be interfacing with a distal portion of the tether. In these aspects, moving the anchor can include compressing at least a portion of the positioning tool and/or tensioning the tether. In these aspects, moving the anchor can include adjusting at least one of a height or an angle of the distal end of the positioning tool such that at least a portion of the anchor is substantially parallel to a plane of an annulus of the native valve. In these aspects, the second position can be nearer to the native valve annulus than the first position. In these aspects, the tracking the delivery device into the first chamber can include inserting a proximal end of the tether into a port positioned at a distal end of the delivery device. In these aspects, the port can be positioned concentric with an outer sheath of the delivery device.

According to some aspects, a method for treating a diseased native valve in a patient includes: concentrically tracking a delivery device that is carrying a prosthetic valve concentrically along a tether into a first chamber of the heart, a distal end of the tether coupled with an anchor near a native valve annulus of the heart; further concentrically tracking the delivery device over the tether into a second chamber of the heart to position the prosthetic valve across the native valve annulus; and exposing the prosthetic valve to deploy a valve prosthesis.

In these aspects, the concentrically tracking can be through a port of a nosecone that is coupled at a distal end of the delivery device. In these aspects, the concentrically tracking can be through a lumen of a valve delivery member that is carrying the prosthetic valve within the delivery device. In these aspects, during the concentrically tracking, a proximal end of the tether can be extending from a portion of the delivery device that is external to the patient.

These and other aspects are described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1G show an embodiment of a method of delivering an anchor for a valve prosthesis near a native valve;

FIGS. 2A-2H show an embodiment of a tether inversion procedure as part of delivering a valve prosthesis;

FIGS. 3A-3J depict an embodiment of a method of delivering a valve prosthesis to an anchor previously placed near a native valve annulus;

FIGS. 4A-4C illustrate various views of embodiments of an interaction between a distal portion of a tether and a distal end of a positioning tool;

FIGS. 5A and 5B show embodiments of a positioning tool and a valve delivery catheter being used to deliver a valve prosthesis; and

FIGS. 6A-6D illustrate a portion of a positioning tool having an arrangement of preferentially bendable regions;

FIG. 7A-7D illustrates a perspective and section views of a prosthetic valve that is carried within a valve delivery catheter;

FIG. 8 is a flow chart of a method of deploying a prosthetic valve using a tether;

FIG. 9 is a flow chart of a method of deploying a prosthetic valve using a tether and a positioning tool;

FIGS. 10A and 10B illustrate a side view and a closeup view of an exemplary positioning tool;

FIGS. 11A and 11B illustrate a side view and a closeup view of another exemplary positioning tool;

FIG. 12 is a flow chart of a method of delivering a valve prosthesis including inverting a tether for tracking the valve prosthesis;

FIGS. 13A-13C show images of an example tether inversion procedure performed within a lamb heart;

FIGS. 14A-14C show images of an example use of a positioning tool in a heart to control axial height of an anchor; and

FIGS. 15A and 15B show images of an example deployment of a prosthetic valve in a heart illustrating issues related to positioning tool length.

### DETAILED DESCRIPTION

Described herein are devices and methods for use in delivering a valve frame and valve, for example during a mitral valve replacement. The devices and methods can be used in conjunction with a previously placed anchor which is used to deliver the valve frame. The devices and methods can be used to transition (e.g., move) the anchor from a first position that is spaced apart from the native valve annulus (e.g., in a sub-annular space) to a second position that is closer to the native valve annulus, prior to deployment of the prosthetic valve.

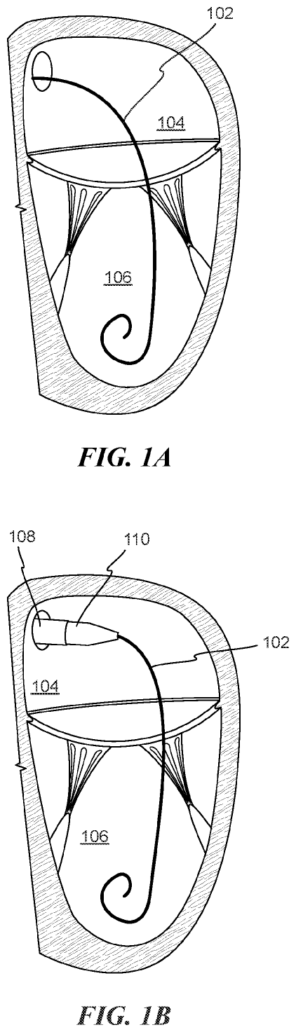

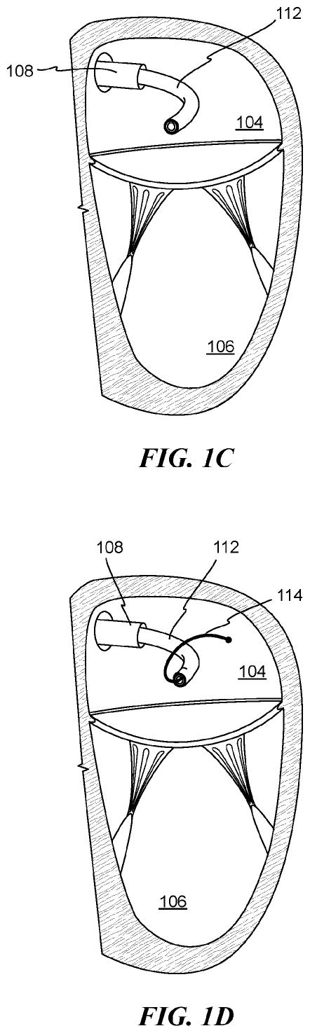

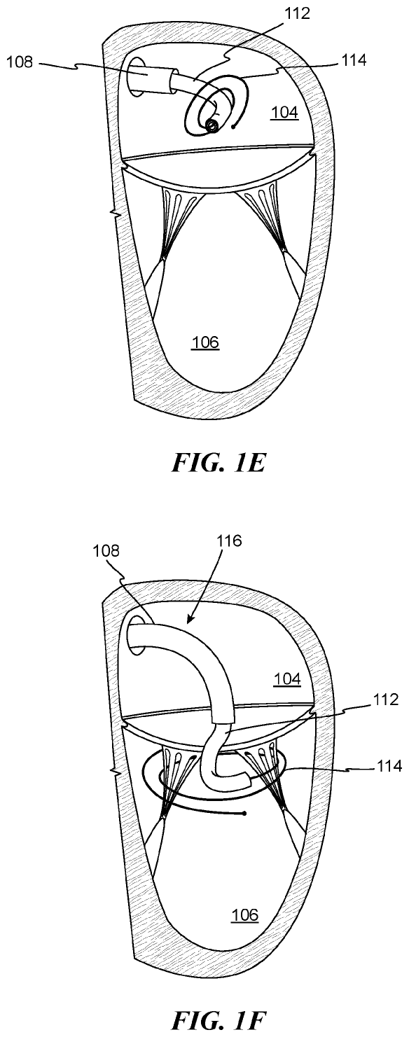

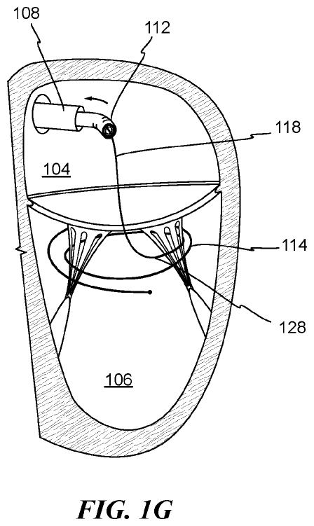

FIGS. 1A-1G show a method of delivering an anchor of a prosthetic valve system using an anchor delivery device or system. At FIG. 1A, a transseptal puncture is made. A guidewire 102 is then routed through the puncture site and positioned either in the left atrium 104 or across the mitral valve into the left ventricle 106. At FIG. 1B, an outer sheath 108 (also referred to as an anchor delivery catheter or steerable catheter) is tracked over the guidewire 102 until the distal end of the outer sheath 108 protrudes into the left atrium 104. In some examples, the outer sheath optionally includes an inner dilator 110. The guidewire 102 and inner dilator 110 (if used) are then removed from the outer sheath 108. At FIG. 1C, an inner sheath having a distally disposed anchor guide 112 is inserted through the outer sheath 108 until the distal tip of the anchor guide 112 extends into the left atrium 104. The anchor guide 112 can be configured to take on a pre-determined curved shape. The anchor guide 112 can be positioned and/or oriented as desired by steering the distal end of the sheath 108 and/or rotating the inner shaft and anchor guide 112 within the sheath 108. In some examples, the distal portion of the outer sheath 108 is bendable between a straight configuration and a bent configuration. Such bending can be controlled, for example, at a handle operationally connected to the anchor delivery catheter. At

FIG. 1D, once the anchor guide 112 is in a selected orientation, the anchor 114 is pushed out through distal tip of the anchor guide 112. The geometry (e.g., curvature) of the anchor guide 112 can cause torsion on the anchor 114 during deployment.

At FIG. 1E, the anchor guide 112 urges the anchor 114 to deploy concentrically with the distal portion of the outer sheath 108 into the atrium 104. At FIG. 1F, the entire delivery system 116 can be pushed and steered (for example, via steering mechanisms in the outer sheath 108) towards an apex of the ventricle 106, crossing through the mitral valve. In some embodiments, counter-rotation of the anchor 114 (via counter-rotation of the inner shaft and guide 112) may aid in advancing the anchor across the mitral valve without entanglement of the chordae. Once the anchor 114 is at a selected depth within the ventricle 106, forward rotation of the anchor 114 (via forward rotation of the inner shaft and guide 112) enables the anchor 114 to encircle the mitral leaflets and chordae. In some embodiments, the anchor 114 is (e.g., initially) deployed towards the apex to aid in avoiding interference with mitral leaflet motion. At FIG. 1G, the outer sheath and inner sheath with anchor guide 112 are removed, leaving a tether 118 that is in place. The tether is still attached to the anchor 114 at attachment 128, and extends through the delivery path and external to the patient. Embodiments of methods and devices for delivering an anchor/valve prosthesis are described in U.S. patent application Ser. No. 16/824,576, filed Mar. 19, 2020 and U.S. patent application Ser. No. 16/594,946, filed Oct. 7, 2019, the entire disclosures of which are incorporated by reference herein.

In some embodiments, the tether that is attached to the anchor can function as a guidewire for a valve delivery device (also referred to as a valve delivery catheter or valve delivery member) to track along for delivering a valve prosthesis. The valve delivery device can include a lumen through which the tether is tracked. In some embodiments, the tether can be inverted or looped within a chamber of the heart, such as the ventricle. For example, the tether can extend from the anchor (e.g., proximate to the mitral annulus) towards the ventricle and can then bend, curve, loop, or invert so as to extend back up through the mitral annulus (e.g., into the atrium). The inversion of the tether can advantageously enable access to the anchor from a sub-annular position. In some examples, the looping of the tether is formed and/or contained within a central opening (e.g., inner radius) of the anchor. A tether that is looped within the central opening of the anchor can enable concentric delivery of a valve over the tether. Further, concentric tracking of the valve delivery device over the tether can advantageously improve its advancement through the patient anatomy. For example, the tip of the valve delivery device can be guided via the concentric tether through a (e.g., pre-existing) puncture in the septum to improve a crossing of the valve delivery device during a transseptal procedure. Further, concentric delivery may reduce the total number of devices for valve delivery since the tether may also be used as a guide wire. Advantageously, concentric tracking of the valve delivery device over the tether can improve a placement of (1) the distal end of the valve delivery device with respect to a chamber of a native heart valve (e.g., left ventricle) and/or (2) a valve prosthesis with respect to an annulus of the native heart valve (e.g., mitral valve). A tether may be formed to comprise one or more of the following materials: stainless steel, nickel titanium alloy (e.g., nitinol), cobalt chromium nickel alloy (e.g., Elgiloy®), cobalt chromium, a polymer, or a block copolymer comprising polyamide and polyether (e.g., Pebax®).

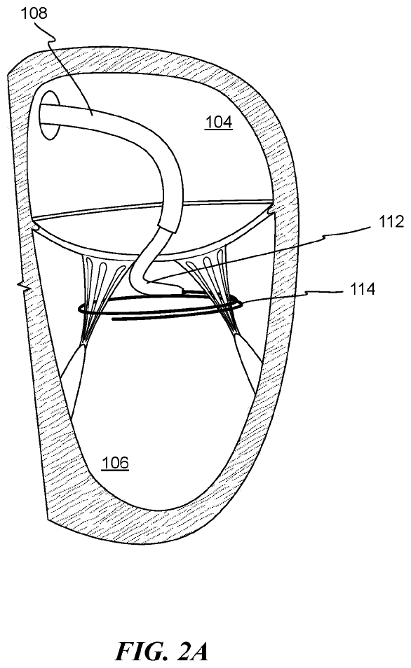

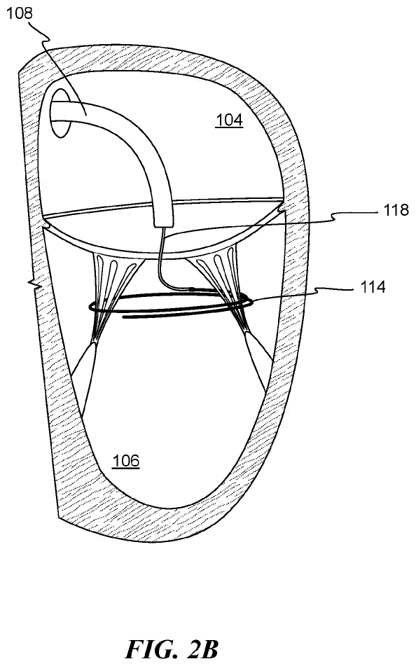

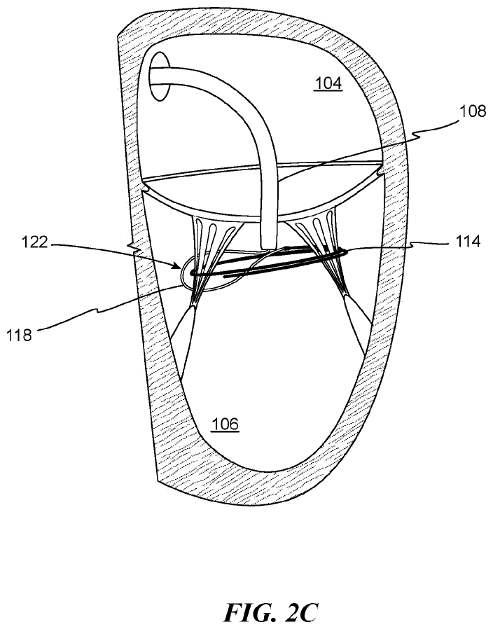

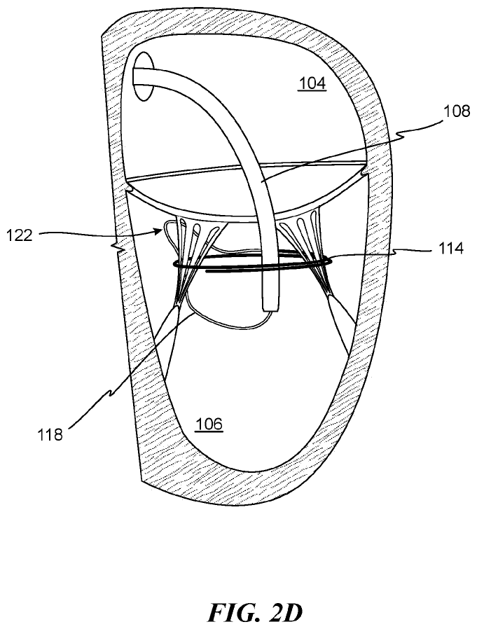

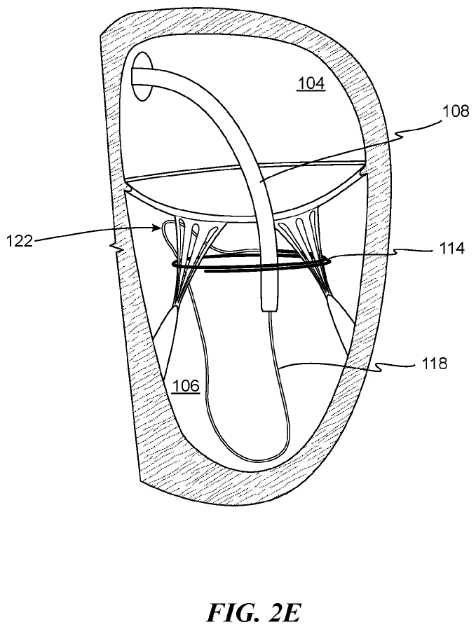

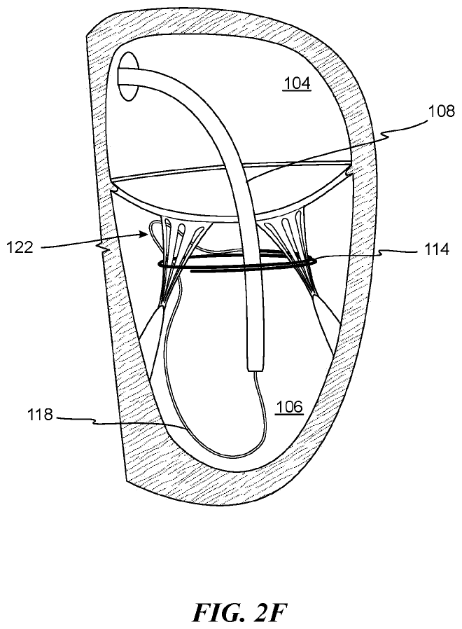

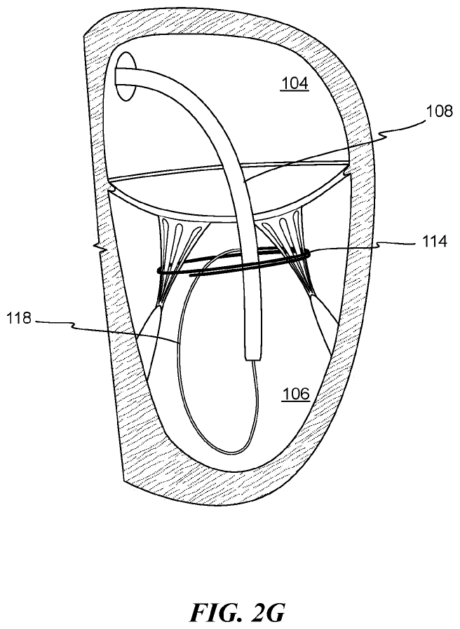

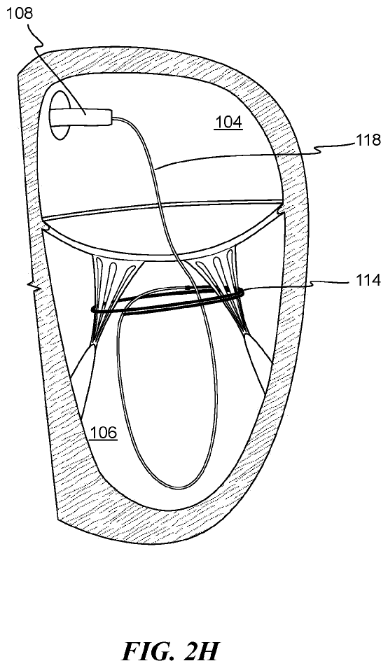

FIGS. 2A-2H show an exemplary method of inverting the tether 118, according to some embodiments, as part of delivering an anchor of a prosthetic valve system within a patient. FIG. 2A shows the anchor guide 112 after being at least partially pushed across the mitral valve plane and used to position the anchor 114 such that the anchor 114 encircles the mitral leaflets and/or chordae (like as shown in FIG. 1F). At FIG. 2B, the distal end of the anchor guide 112 is retracted proximally into the outer sheath 108 and the tether 118 is exposed from the distal end of the outer sheath 108. The tether 118 can be configured to be sufficiently flexible to bend laterally as it traverses the patient's vessels and heart, but also be sufficiently stiff to resist becoming tangled as it is manipulated once out of the outer sheath 108. The stiffness of the tether 118 may provide some resistance when attempting to feed the tether 118 through a central opening of the anchor 114.

The steerable distal tip of the outer sheath 108 by be used to advance a portion of the tether 118 through the anchor 114. For example, at FIG. 2C, the outer sheath 108 is advanced through the native valve annulus (e.g., coaxially) using the steering mechanism of the outer sheath 108. In addition, the tether 118 can be fed distally through the outer sheath 108 to provide a slack 122 of tether 118. In general, a tether that contains slack may include a condition of the tether in which an incremental (e.g., small) proximal retraction of the tether 118 serves to reduce the length of the tether 118 that is exposed from the outer sheath 108, without application of substantial force to the anchor 114. In this context, a substantial force is a force of sufficient magnitude to alter a position of the anchor 114 with respect to the native anatomy. Slack within the tether 118 may cause a portion of the tether 118 to form a coil or loop within the atrium 104 and/or ventricle 106. While the tether slack and loops/coils form within a ventricle of the heart depicted in the example of FIGS. 2A-2H, it will be appreciated that other examples of the present disclosure alternatively or additionally include tether slack and loops/coils that are formed in an atrium.

At FIG. 2D, the distal end of the outer sheath 108 is advanced further through the central opening of the annular-shaped anchor 114, and across the plane of the anchor 114, to allow a loop of the tether 118 to enter through the central opening of the anchor 114 and into the left ventricle 106. At FIG. 2E, the tether 118 is further fed through the outer sheath 108 and deployed into the left ventricle 106. At FIG. 2F, the outer sheath 108 is advanced further toward the left ventricle apex. In some cases, the tether 118 may become constrained within the anchor 114 due to tension being put on the tether 118 as it is advanced through the annulus of the anchor 114. At FIG. 2G, the tether 118 is retracted proximally with respect to the outer sheath 108 enough to remove at least a portion of the slack 122 of the tether 114 remaining within the left ventricle 106, while still leaving enough of the length of the tether 118 within the ventricle 106 to enable access to the anchor 114 from a sub-annular position with respect to the anchor 114 during delivery of the prosthetic valve. As shown, this can allow the tether 118 to unwind from a constrained/coiled configuration to an inverted configuration, where the tether 118 reverses its orientation and the sub-annular portion of the tether 118 takes on a U-shaped bend. At FIG. 2H, the outer sheath 108 is retracted proximally leaving the tether 118 in the inverted position within the left ventricle 106 for subsequent concentric delivery of the prosthetic valve over the tether 118.

While the procedure shown in FIGS. 2A-2H is presented as a sequence of operations, it will be appreciated that one or more of the operations can occur in a different sequence. For example, the tether 118 may be positioned within the ventricle 106 in the inverted configuration using a number of different combinations of advancing/retracting the outer sheath 108 toward/away from the anchor 114, and advancing/retracting the tether 118 with respect to the outer sheath 108.

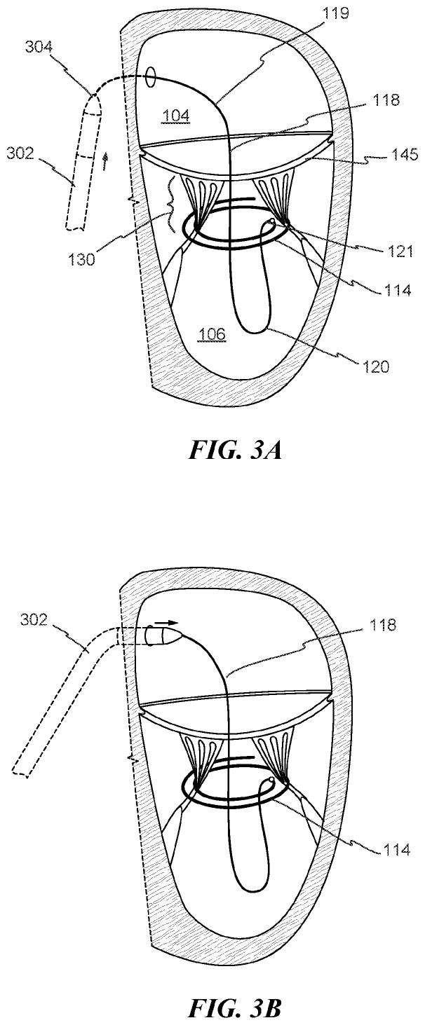

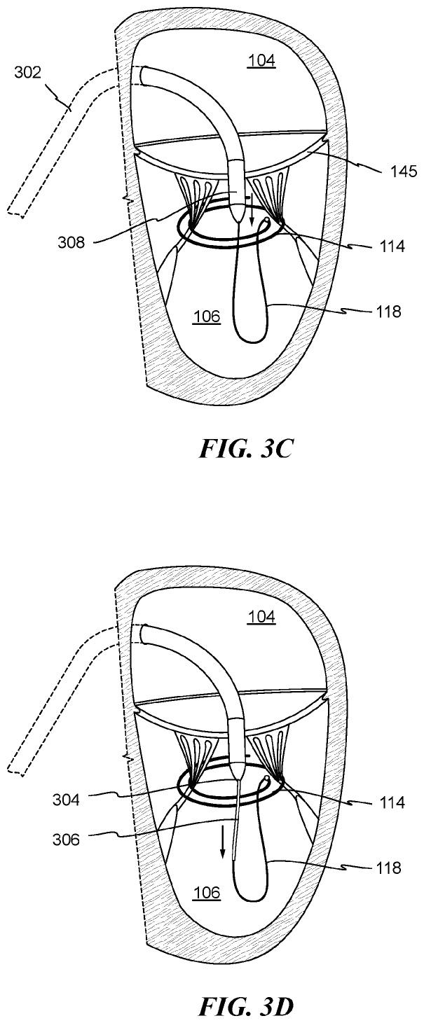

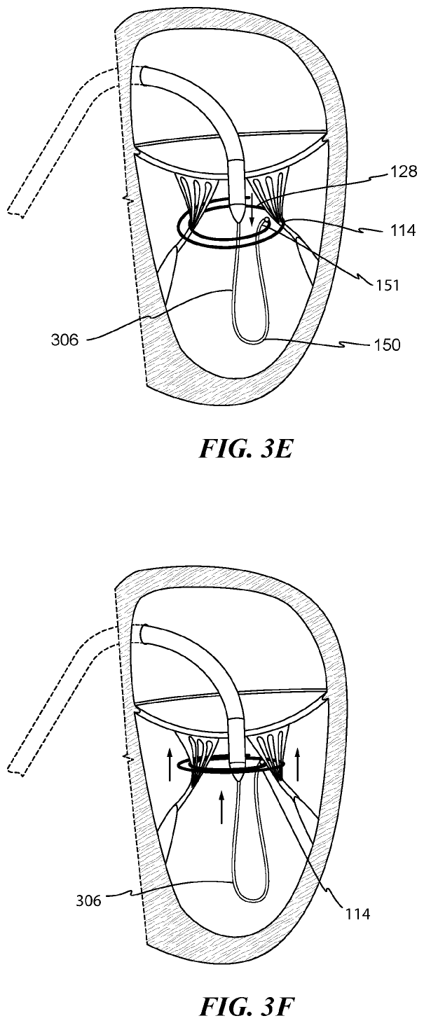

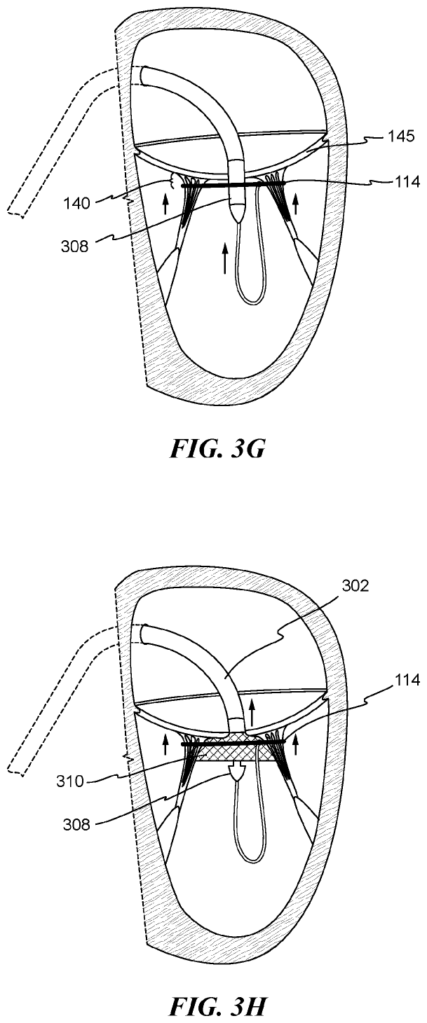

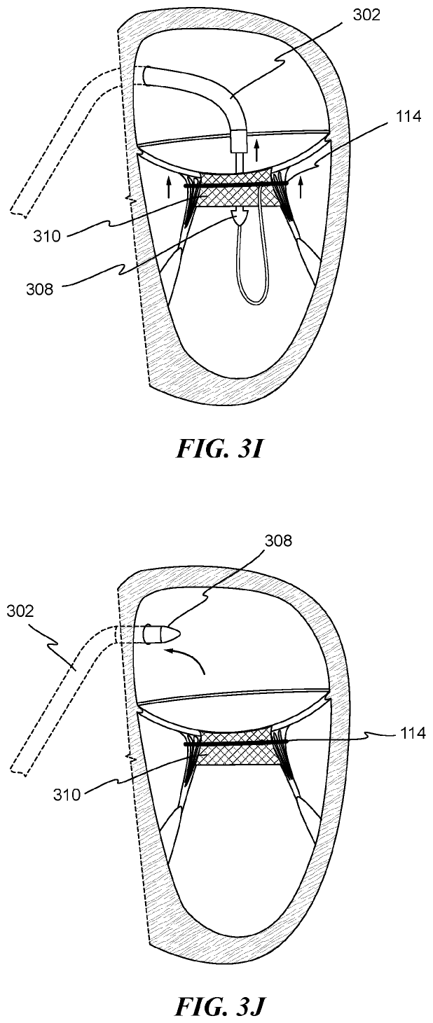

FIGS. 3A-3J show an embodiment of delivering a valve after an anchor has already been placed by an anchor delivery device or system (e.g., as shown in FIG. 2H). The tether 118 can be attached to an anchor 114 that at least partially encircles chordae and/or leaflets of the native valve. The tether 118 can function as a guidewire for a valve delivery device.

As described above, in some embodiments, prior to and/or during delivery of valve prosthesis, the tether 118 can be positioned from a first configuration (e.g., as in FIG. 2F) to a second configuration (e.g., as in FIG. 2G) using the anchor delivery catheter. In some embodiments, the tether 118 in the second configuration includes at least one bend that inverts or substantially reverses the orientation of the tether 118 (i.e., the tether 118 can include a “U-bend”). In some embodiments, the U-bend or inversion of the tether 118 can be developed near or adjacent an apex of the heart (e.g., FIG. 3A, 120). In some embodiments, the inverted configuration of the tether 118 can be such that a middle axial portion of the tether 118 is relatively centrally located with respect to the native valve annulus 145, leaflets, and/or chordae while the distal end of the tether remains attached to the anchor 114. In some embodiments, the one or more bends of the tether 118 enable the tether 118 to maintain coupling with the anchor (e.g., FIG. 3A, 121) while providing a (e.g., substantially) concentric tracking path for the distal tip of the valve delivery catheter 302 to extend into the second chamber 106 and to align the waist of the valve prosthesis with the native valve annulus 145. In some embodiments, the second configuration (i.e., the inverted configuration) of the tether 118 can be such that the valve delivery catheter 302 is able to be (e.g., concentrically) tracked along the tether 118 from the first chamber 104, through the valve annulus, and into the second chamber 106. In other embodiments, the one or more bends of the tether 118 enable the tether 118 to maintain coupling with the anchor 114 when the proximal end of the anchor 114 points into the ventricle.

As shown in FIG. 3A, the tether 118 can have sufficient length such that a proximal portion is external to the patient, a middle portion is traversing the vasculature of the patient, and a distal portion is attached to the anchor 114. Additionally, the middle portion can extend through to the transseptal puncture, into a first chamber of the heart (e.g., the atrium), across a heart valve (e.g., the mitral valve), and then loop or invert (i.e., reverse direction) in a U-bend within a second chamber of the heart (e.g., the ventricle). In some embodiments, and as shown in FIG. 3A, the tether 118 can be advanced so as to bend in both the first chamber 104 (e.g., bend 119) and the second chamber 106 of the heart (e.g., bends 120, 121). In some embodiments, at least two bends of the tether 118 comprise different bending angles. In some embodiments, at least two bends of the tether 118 have a different radius of curvature. In some embodiments, at least two bends of the tether 118 comprise bending angles that are substantially the same. In some embodiments, at least two bends of the tether 118 have a radius of curvature that is substantially the same. In some embodiments, the one or more tether bends are developed in a sub-annular space (e.g., within the left ventricle). In some embodiments, one or more bends of the tether 118 contact a portion of the inner wall of the first chamber 104 and/or the second chamber 106. Contact of a portion of the tether with a wall of the heart chamber may promote formation of one or more bends in the tether 118. The contact with a portion of the inner wall can be for a selected (e.g., predetermined) time, as controlled by an operator of the valve delivery catheter.

In some embodiments, the tether 118 can be maintained to be substantially free of tension (prior to and/or during the tracking of the valve delivery catheter 302 thereover). The tether 118 may be formed such that, when pushed (e.g., for delivery thereof or tracking of a valve delivery catheter 302 thereover), it is sufficiently resistant to kinking so as to be advanced within the patient anatomy and/or through or along the valve delivery catheter 302. In some embodiments, the tether 118 is sufficiently flexible so as to develop curvature (e.g., the one or more bends as described herein) when pushed.

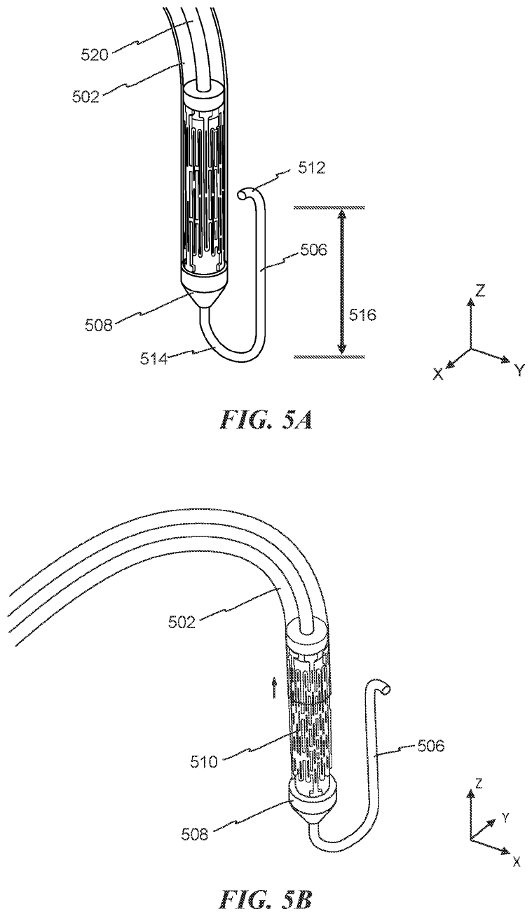

As shown in FIG. 3B, a valve delivery catheter 302 can be tracked over the tether 118, through the transseptal puncture into the first chamber 104 of the heart. In one embodiment, the valve delivery catheter 302 can be a concentric delivery catheter. In this embodiment, the valve delivery catheter 302 can have a concentric port 304 for tracking over the tether 118 (i.e., the port 304 can include a central point or axis that is concentric with a central axis of the valve delivery catheter 302). The port 304 may be in a nosecone, which is a conical portion a the distal end of the valve delivery catheter 302. The valve delivery catheter 302 can have an inner hollow shaft (“inner shaft”) forming a lumen that is shaped and sized for receiving the tether 118 (e.g., see FIG. 5A, 520). The inner shaft that forms the lumen of the valve delivery catheter 302 can be substantially centrally located, e.g., along a central axis of the valve delivery catheter 302. The prosthetic valve 510 can be compressed within a space between the inner shaft and the outer sheath of the valve delivery catheter 302. Thus, the inner shaft of the valve delivery catheter 302 can run through the central opening of the prosthetic valve 510. The port 304 can be connected with the inner shaft that forms the lumen of the valve delivery catheter 302. The port 304 can be located at a distal end (e.g., tip) of the valve delivery catheter 302. In some embodiments, the tether 118 is loaded into the port 304 while the valve delivery catheter 302 is external to the patient, and the valve delivery catheter 302 is subsequently tracked over the tether 118. The tether 118 can extend from the transseptal puncture through a first chamber of the heart 104 (e.g., the left atrium), through a valve annulus (e.g., the mitral valve) to a second chamber of the heart 106 (e.g., the left ventricle).

FIG. 3C shows the exemplary concentric valve delivery catheter 302 tracked over the tether 118 until a valve delivery member 308 (e.g., a valve capsule) that carries a valve prosthesis is positioned across the native valve annulus 145. In some embodiments, the valve prosthesis is retained within the valve delivery catheter 302 directly, without use of a valve delivery member 308. For example, the valve prosthesis 302 can be fitted within the valve delivery catheter 302 at a distal portion thereof, in a collapsed state. In some embodiments, the valve prosthesis is coupled to a nosecone of the valve delivery catheter 302 and is deployed by separation of the nosecone and the distal end of the valve delivery catheter. In some embodiments, the valve prosthesis is deployed by use of a pusher catheter to move the valve prosthesis distally beyond the distal end of the valve delivery catheter 302. With the valve delivery member 308 (or distal end of the delivery catheter 302) positioned across the native valve annulus, a majority (e.g., all) of the tether 118 that extends from the port 304 of the valve delivery catheter 302 to the anchor 114 is in the second chamber 106 (e.g., left ventricle). In some embodiments, the anchor 114 remains in or near the first spaced apart position 130 while the valve delivery catheter 302 is tracked over the tether 118 to cross the native valve annulus.

In some embodiments, the anchor 114 can be in a first position 130 that is relatively spaced apart from the valve annulus during the tracking of the valve delivery catheter 302 over the tether 118. In the first spaced apart position 130, the anchor 114 is sufficiently removed from the valve annulus to minimize interference between the anchor 114 and the motion of the native valve leaflets and/or the chordae. The first spaced apart position 130 of the anchor 114 with respect to the valve annulus can reduce occurrence of paravalvular leakage (PVL) attributed to interference of the anchor with the native valve leaflets and/or the chordae, for example, prior to deployment of the valve prosthesis. In some embodiments, the anchor 114 in the first spaced apart position 130 is from about 10 millimeters (mm) to about 40 mm away with respect to the annulus of the native valve, in an apical direction.

At times, it may be advantageous to move the anchor 114 from the first (spaced apart) position to a second position closer to or near the native valve annulus 145 after tracking of the valve delivery device over the tether (e.g., prior to or during deployment of the prosthetic valve). Having the anchor 114 positioned in the closer position (e.g., adjacent or abutting the native valve annulus 145) can facilitate sealing of the prosthetic valve frame and anchor 114 around the mitral leaflet bodies and/or the chordae. This sealing may substantially reduce or prevent PVL for the deployed prosthetic valve. In some embodiments, the anchor 114 can be moved from the first spaced apart position 130 to the second closer position 140 using the (e.g., tensioned) tether and/or a (e.g., stiffened) positioning tool as described further herein. In some embodiments, the second position 140 can be less than from about 3 mm to about 0.5 mm. In some embodiments, the second position 140 can be contacting at least a portion of the native valve annulus 145. Movement of the anchor 114 can result in a change in the anchor height (e.g., superiorly and/or inferiorly) and/or the anchor angle with respect to the heart anatomy (i.e., an angle that extends between a plane that substantially includes the anchor and a native valve (e.g., annular) plane).

In some embodiments, a positioning tool can be used to adjust a position of the anchor 114 after the tether 78 has been inverted. FIG. 3D shows an example of a positioning tool 306 being tracked distally along the inverted tether 118 after the valve delivery catheter 302 has been positioned across the native valve annulus. In some embodiments, the positioning tool 306 can be, or include, a hollow elongate body and have sufficient lubricity, shape, and size to fit and track over the tether 118 within the lumens of the valve delivery catheter 302 and/or the valve delivery member 308. The positioning tool 306 may be sufficiently flexible to track over and take on the curvature of the tether 118. The positioning tool 306 can be configured to bend and rotate as it traverses the tether 118. The positioning tool 306 can conform to the variable curvatures the tether 118 may take in the left ventricle 106. The flexibility of the positioning tool 306 during tracking can reduce the chances that the positioning tool 306 will interact with and/or load the tether 118 and cause the anchor 114 to rotate or de-encircle. A proximal portion of the positioning tool 306 can extend external to the patient and be fitted to a control of the valve delivery catheter 302 that enables manipulation thereof. The control may include one or more actuators (e.g., button, knob, turnable member and/or switch), for example, of a handle at the proximal end of the valve delivery catheter 302. Manipulation of the positioning tool 306 can comprise translation, rotation, and/or compression. The positioning tool 306 can be distally advanced over the tether 118 until a distal end of the positioning tool 306 communicates (e.g., interfaces) with a distal portion of the tether 118 and/or proximal end of the anchor 114. In some cases, the distal end of the positioning tool 306 may communicate (e.g., interface) with an attachment that releasably attaches a proximal end of the anchor 114 with the distal end of the tether 118. The positioning tool can be formed of any material or combination of materials as are used to form the tether.

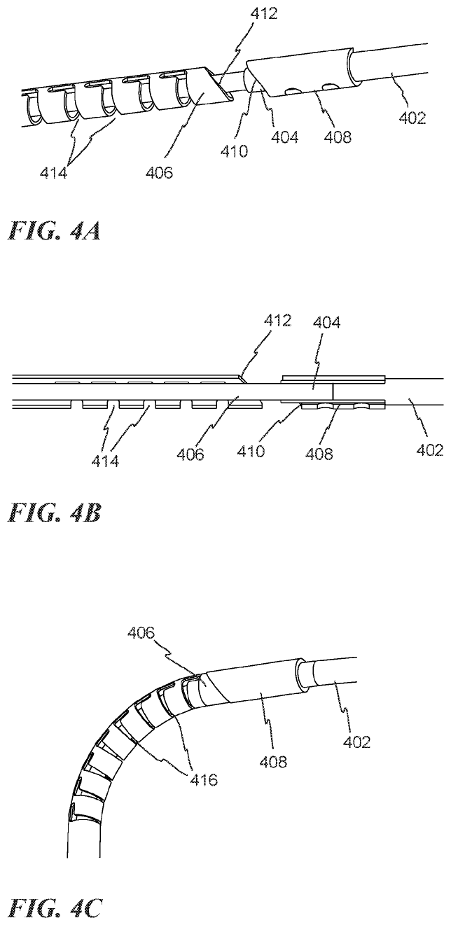

Compression of the positioning tool 306 axially can be used for stiffening the positioning tool 306 and/or for developing predetermined curvature and/or bends at selected portions of the positioning tool (e.g., and encompassed tether). See, for example, FIGS. 4A-4C.

The selected portions can be regions that are preferentially bendable (see, for example, FIGS. 4A-4C). In some embodiments, stiffening the positioning tool 306 urges the tether 118 to assume a predetermined shape and/or configuration. Bending/compression/stiffening of the positioning tool 306 may be activated, for example, by pushing the positioning tool 306 relative to the tether 118 and/or pulling the tether 118 relative to the positioning tool 306. In some examples, this compressive force may be controlled by the one or more actuators of the valve delivery catheter 302. In some cases, actuation of the positioning tool 306 may be controlled by a handle/system separate from the valve delivery catheter 302. The one or more actuators may be configured to provide a controlled degree of tension on the positioning tool 306. In some cases, the one or more actuators may be configured to lock the positioning tool 306 in the stiffened and bent configuration.

FIG. 3E shows an example of the positioning tool 306 having been tracked over the tether 118 until a distal portion thereof has interfaced with a distal portion of the tether 118 at an interface region 128. The positioning tool 306 has been inverted and compressed/stiffened to a predetermined shape. Thus, the positioning tool 306 has taken on one or more predetermined bends at 150 (e.g., U-bend, toward an apex of the heart) and 151 (adjacent to the attachment with the anchor 114). The stiffened positioning tool 306 can additionally be used to tension the tether 118 (e.g., via interaction of the tether 118 and the positioning tool 306 at the interface region) to enable precise control over movement of the anchor 114.

FIG. 3F shows an example of using a stiffened (e.g., compressed) positioning tool 306 to move an anchor 114 from a first position (e.g., FIG. 3A, 130) toward a second position that is nearer to a native valve annulus (e.g., FIG. 3G, 140). Alternatively or additionally, the tether 118 can be tensioned to move the anchor 114. In one embodiment, the stiffened inverted positioning tool 306 can be used to achieve axial alignment between the anchor 114 and the valve delivery catheter 302. Alignment may include aligning the anchor 114 to sit in a plane that is perpendicular to the plane of the distal end of the valve delivery catheter 302. The stiffened inverted positioning tool 306 can also be used to move the anchor 114 up (proximally), as shown in FIGS. 3F and 3G, toward valve annulus to better engage with valve leaflets and reduce leakage around valve/anchor (paravalvular leakage). In some embodiments, the stiffened inverted positioning tool 306 enables the anchor 114 to achieve planarity with the valve annulus 145, which helps ensure planar positioning of the valve frame with respect to the mitral annulus. The positioning tool 306 can also ensure that the anchor 114 maintains good encircling of the native anatomy (e.g., chordae and leaflets).

FIG. 3G shows an example of the anchor 114 in a (e.g., second) position 140 that is near the native valve annulus, in preparation for deployment of a prosthetic valve from a valve delivery member 308 of the valve delivery catheter 302. In some embodiments, the positioning of the anchor 114 may be assessed prior to and/or during deployment of the prosthetic valve. The positioning tool 306 and/or tether 118 can be used to make any selected adjustments to the position of the anchor 114, as deemed necessary by a clinician.

FIG. 3H shows an example of a first portion of a prosthetic valve deployment from the valve delivery member 308 of concentric valve delivery catheter 302. The first portion of deployment can be a partial deployment of the prosthetic valve. In the example of FIG. 3H, the outer sheath of the valve delivery catheter 302 is retracted proximally to expose a distal portion of the valve deliver member 308 that carries the prosthetic valve 310. In some embodiments, the prosthetic valve 310 is self-expanding. Upon retraction of the retaining outer sheath, the prosthetic valve 310 can expand to contact the native leaflets and/or chordae that are encircled by the anchor 114. In some embodiments, the first portion of the prosthetic valve 310 that is deployed includes portions of the prosthetic valve 310 that are in the sub-annular space, or in the second chamber 106 of the heart. In some embodiments, the first portion of the prosthetic valve 310 that is deployed includes portions of the prosthetic valve 310 that are in the super-annular space, or in the first chamber 104 of the heart.

FIG. 31 shows an example of continued deployment of a second portion of the prosthetic valve 310 from the valve delivery member 308. The continued deployment of the prosthetic valve 310 can include further proximal retraction of the valve delivery catheter 302. The continued deployment of the prosthetic valve 310 can include expansion of remainder of the prosthetic valve 310. In some embodiments, the second portion includes the remaining portion of the prosthetic valve 310 that was not deployed during the deployment of the first portion. In some embodiments, the second portion of the prosthetic valve 310 that is deployed includes portions of the prosthetic valve 310 that are in the super-annular space, or in the first chamber 104 of the heart. In some embodiments, the second portion of the prosthetic valve that is deployed includes portions of the prosthetic valve that are in the sub-annular space, or in the second chamber 106 of the heart. The valve 310 can be held in place using a compression fit between the valve 310 and the anchor 114. As shown in FIG. 3H, the valve 310 may comprise one or more flares, for example a ventricular flare and an atrial flare. The prosthetic valve can include a waist that forms a midsection of the valve 310. The valve 310 may seat against the anchor 114 such that the anchor surrounds the waist section of the valve 310.

While the foregoing describes deployment of the prosthetic valve 310 by proximal retraction of the concentric valve delivery catheter 302, it will be appreciated that alternative modes of deployment of the prosthetic valve 310 are possible. For example, in some embodiments the valve delivery member 308 can be distally advanced from its (e.g., initial) position across the mitral valve to at least partially expose a first portion of the prosthetic valve 310. Alternatively or additionally, the valve delivery catheter 302 can be proximally retracted to at least partially expose a second portion of the prosthetic valve. In some embodiments, the first portion is a distal portion and the second portion is a proximal portion of the prosthetic valve 310. In some embodiments, the first portion is a proximal portion and the second portion is a distal portion of the prosthetic valve.

Additionally, while the foregoing describes deployment of the valve from a concentric delivery catheter, it should be understood that alternative modes of deployment of the prosthetic valve 310 with the inverted tether and/or positioning tool are possible. For example, the valve delivery catheter can include a monorail lumen for the tether. Exemplary valve delivery systems that can be used with an inverted tether and/or positioning tool as described herein are described in PCT Application No. PCT/US2021/026463, titled “VALVE DELIVERY SYSTEM,” and filed on Apr. 8, 2021, the entirety of which is incorporated by reference herein.

In some embodiments, the inverted positioning tool 306 and/or tether 118 may be used to adjust a position of the anchor 114 prior to, during, and/or following deployment of a some or all of the prosthetic valve 310. Adjustment of the position may be made in response to a clinician assessment of performance of the native valve leaflets, chordae, hemodynamics, and/or of the prosthetic valve. Once the prosthetic valve 310 is fully deployed and position is configured, the positioning tool 306 may be retracted, and the tether 118 may be released from the anchor 114 and withdrawn. In some embodiments, the tether 118 can be detached from the anchor via a release coupling. Exemplary releasable couplings are described in U.S. patent application Ser. No. 16/824,576, the entirety of which is incorporated by reference herein. The valve delivery catheter 302 and valve delivery member 308 can likewise be withdrawn and removed. FIG. 3J shows the valve delivery catheter 302, including the valve member 308, being withdrawn and removed from the body, leaving the anchor 114 and prosthetic valve 310 in place.

FIGS. 4A-4C show an exemplary manner of interaction between a proximal end of an anchor, a distal portion of a tether, and a distal end of a positioning tool (e.g., during deployment of the anchor and tether as described with respect to FIGS. 3A-3J). FIGS. 4A and 4B show perspective and side cross-sectional views, respectively, of a proximal end of an anchor 402, a distal portion of a tether 404, and a distal end of a positioning tool 406. As shown in FIG. 4A, the proximal end of the anchor 402 is attached to the tether 404 at a releasable attachment 408. The attachment can comprise bond joint, a solder joint, a crimp, or a fastener. In some embodiments, the tether 404 and anchor 402 are attached prior to delivery of the anchor 402. In some embodiments, the attachment comprises at least one end having a selected geometry that is sized and shaped for interaction with the positioning tool 406. In the example of FIGS. 4A-4C, the attachment 408 comprises a wedge portion having an angled or beveled proximal end 410 generally shaped to interact or mate with an angled or beveled distal end 412 of the positioning tool 406. As the positioning tool 406 is advanced distally along the tether 404, it eventually encounters the wedge portion 408 of the tether 404. The interaction between the wedge portion 408 and the positioning tool 406 can create aligned, torsionally stiff interaction between the tether 404 (and attached anchor 402) and the positioning tool 406. It will be appreciated that other shapes for the distal portion 410 of the tether and the distal end 412 of the positioning tool are also possible, so long as the ends are shaped to interact with one another in a torsionally stiff manner. For example, the distal end 412 of the of the positioning tool 406 can be a straight-cut edge (e.g., perpendicular to the axis of the positioning tool 406 when in a straight configuration).

The positioning tool 406 can comprise spaces, gaps, or cutouts (e.g., 414) along at one or more distal portions. Under compression in an axial direction, the cutouts promote preferential flexion of the positioning tool in selected (e.g., predetermined) directions and/or angles. FIG. 4C depicts an example of compression of the positioning tool 406, where the distal end of the positioning tool 406 has been advanced to interface against the attachment 408, and in which cutouts 416 have developed a reduced gap width under the compression. Compression of the positioning tool 406 can cause one or more portions to develop a curve (e.g., turn, or bend). An amount of developed curvature can depend upon a geometry of the cutouts, and/or an amount of applied compression to the positioning tool. The interaction or mating is such that relative rotation between the tether 404 and the positioning tool 406 is substantially prevented. Rotation of the distal end of the positioning tool 406, when coupled with the attachment at the tether 404, generates rotation at the distal end of the tether 404 and promotes a rotation of the anchor 402. Rotation of the anchor 402 can be used to adjust an angle at which the anchor 402 is positioned with respect to the chordae, leaflets, and/or annulus. Positioning tools and tethers are further described in PCT/US2021/026463, titled “VALVE DELIVERY SYSTEM,” and filed Apr. 8, 2021, the entirety of which is incorporated by reference herein.

In some embodiments, a plurality of curvatures or bends can form in the inverted stiffened positioning tool along different circumferential aspects or planes of the positioning tool. FIG. 5A shows in perspective view an example of a distal portion of a valve delivery catheter 502 that carries a valve delivery member 508, with a positioning tool 506 extending through lumens formed by inner shafts and/or sheaths therein. FIG. 5B depicts in a different perspective view a portion of a prosthetic valve 510 that is carried by the valve delivery member 508 having been exposed by proximal retraction of the valve delivery catheter 502. For clarity, omitted from these figures are the tether over which the positioning tool is tracked and the anchor for the prosthetic valve to which the tether is attached. The example positioning tool of FIGS. 5A and 5B has a first bend 514 in a Y-Z plane, and a second bend 512 in an X-Y plane when the positioning tool is in the compressed/stiffened state. In some embodiments the compressed positioning tool comprises a bend from about 5° to about 310°. The bend(s) of the positioning tool 506 can align with and/or urge the tether to maintain its configuration (e.g., bends) of being coupled to the anchor, and enabling positioning of the valve delivery catheter 502 and deployment of the prosthetic valve 510. In some embodiments, the compressed positioning tool 506 comprises a bend from about 70° to about 100°, for example about 90°, as shown in FIG. 5A at bend 512. In some embodiments the positioning tool 506 comprises a bend from about 150° to about 310°, for example about 180° as shown in FIG. 5A at bend 514. In some embodiments, the bend comprises a radius of curvature of about 2 millimeters (mm) to about 20 mm. In some embodiments, the positioning tool 506 has a substantially straight portion that is between a first bend and a second bend (e.g., FIG. 5A). The substantially straight portion can have a length from about 10 mm to about 35 mm.

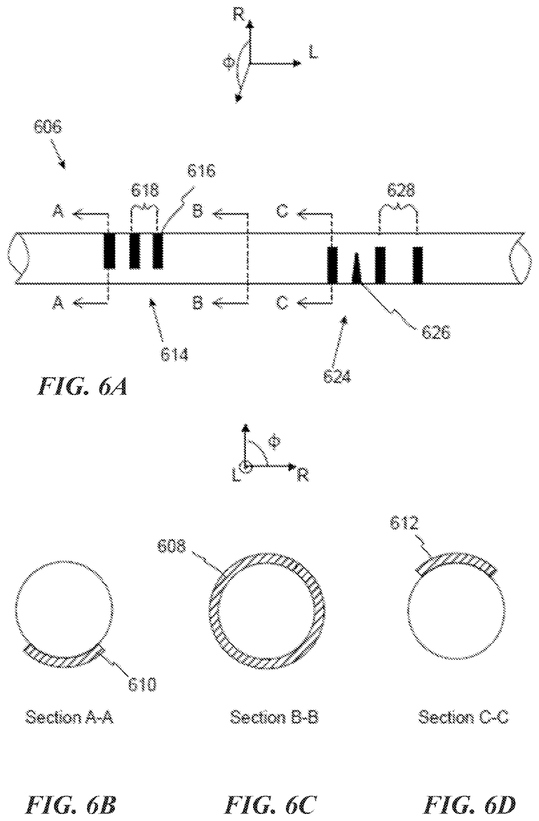

FIGS. 6A-6D illustrate a portion of a positioning tool 606 having an arrangement of preferentially bendable regions intended to enable inversion of the positioning tool 606. The positioning tool 606 is depicted with polar coordinate system having longitudinal, radial, and azimuthal (e.g., angular) axes L, R, and ϕ. Further, the preferentially bendable regions enable the positioning tool 606, during use, to (1) take on a stiffened configuration capable of positioning the anchor in the heart, and (2) develop sections of curvature (bends) that provide space to position the valve delivery catheter and deploy the prosthetic valve. The positioning tool 606 includes an elongate body having an annular wall of generally constant cross-section (e.g., section B-B, 608, FIG. 6C), enabling translation and rotation within the valve delivery catheter. In some embodiments, the preferentially bendable region comprises one or more cutouts that at least partially circumscribe an outer wall of the positioning tool 606. In some embodiments, the cutouts have a generally constant width (e.g., 616, FIG. 6A). In some embodiments, the cutouts have a varying width (e.g., 626, FIG. 6A). In some embodiments, the spacing between adjacent cutouts in a bendable region is generally constant (e.g., 618, FIG. 6A). In some embodiments, the spacing between adjacent cutouts in a bendable region is variable (e.g., 628, FIG. 6A). In some embodiments, a first bendable region (e.g., 614, FIG. 6A) is spaced apart from a second bendable region (e.g., 624, FIG. 6A) along a longitudinal axis of the positioning tool. In some embodiments, a first bendable region (e.g., section A-A, 610, FIG. 6B) is spaced apart from a second bendable region (e.g., section C-C, 612, FIG. 6D) along an azimuthal axis of the positioning tool 606. It should be appreciated that the various shapes, sizes, and placements of the cutouts shown in FIG. 6A are exemplary only and need not all be included (e.g., one positioning tool may include only a portion of the variations shown in the positioning tool 606). Further, while the foregoing describes cutouts as forming the preferentially bendable regions, it will be appreciated by those of skill in the art that a number of techniques may be used for forming preferentially bendable regions in the positioning tool. For example, a braiding pattern that reinforces the outer wall of the positioning tool may comprise regions of reduced reinforcement. For example, the positioning tool may comprise a variable durometer polymeric material, or one or more mechanical hinges.

In any of the embodiments described herein, the positioning tool 306 may be configured to track over the looped and inverted tether 118 without causing de-encircling of the anchor 114. The positioning tool 306 may be configured to raise the anchor 114 axially closer to a plane of the native valve within the sub-annular space. The positioning tool 306 may be configured to support the anchor 114 in position without affecting deployment of the valve prosthesis 310. The positioning tool 306 may be configured to force the valve delivery catheter 302 to track (e.g., concentrically) through the anchor 114.

FIG. 10A shows an exemplary positioning tool 1006 positioned over an inverted tether and engaged with an anchor 1014. The positioning tool 1006 can include a first preferentially bendable region 1044 and a second preferentially bendable region 1046. The first preferentially bendable region 1044 can be configured to bend in along a first plane (e.g., Y-Z plane) and the second preferentially bendable region 1046 can be configured to bend in along a second plane (e.g., X-Y plane) that is different than the first plane. In some examples, the second plane is orthogonal to the first plane. In some examples, the second plane is non-orthogonal to the first plane. The first preferentially bendable region 1044 can be configured take on a U-shaped bend (e.g., positioned toward the apex of the heart). The second preferentially bendable region 1046 can be positioned closer to the distal end 1050 of the positioning tool 1006 compared to the first preferentially bendable region 1044, and can be configured to bend radially inward toward the center of the anchor 1014. Each of the first preferentially bendable region 1044 and the second preferentially bendable region 1046 can include spaces or gaps (e.g., cutouts) that allow these sections to preferentially bend from a straight configuration to a pre-determined shape when a compressive force is applied to the positioning tool 1006 (e.g., as described above with respect to FIGS. 4A-4C). For example, one side of the first preferentially bendable region 1044 can include cutouts that are configured to form a U-shape when activated to a stiffened state. This shape can enable the positioning tool 1006 to support and/or push the anchor closer toward the native valve annulus. One side of the second preferentially bendable region 1046 can include cutouts that are configured to bend the positioning tool 1006 radially inward by a pre-determined angle.

The positioning tool 1006 can have a first section 1030 and a second section 1040. The first section 1030 is between the anchor plane 1032 and a plane 1034 where the curvature of the first preferentially bendable region 1044 begins. The second section 1040 is between the plane 1034 and where the curvature of the second preferentially bendable region 1046 begins. The second section 1040 may be substantially parallel to the first section 1130. An axial length

Li of the first section 1030 may be chosen to hold the anchor 1014 high enough for axial alignment with the valve delivery catheter (and the prosthetic valve) during deployment of the prosthetic valve and provide room for deployment of the prosthetic valve. In some examples, the length Li ranges between any two of the following values: 15 mm, 20 mm, 25 mm, 30 mm, and 40 mm.

FIG. 10B shows a closeup view of a distal portion of the positioning tool 1006. As shown, the distal end 1050 can include a beveled edge that can be shaped to interact or mate with a correspondingly beveled edge of a portion of the tether and/or anchor (e.g., attachment 408). FIG. 10B shows circumferential cutouts 1054 on one side of the second preferentially bendable region 1046 to enable bending to the pre-determined shape (e.g., arc shaped second preferentially bendable region 1046 as shown in FIG. 10A) when an axial compressive force is applied to the positioning tool 1006. For example, the second preferentially bendable region 1046 can be configured to bend toward the cutouts 1054 such that the gap formed of the cutouts 1054 is reduced. Although not shown in FIG. 10B, the first preferentially bendable region 1044 can also include circumferential cutouts to form the pre-determined U-shaped shown in FIG. 10A.

As shown in FIG. 10B, the second section 1040 of the positioning tool 1006 can also include cutouts 1066. The pattern of the cutouts 1066 can be configured to allow the second section 1040 to lateral flex/bend when tracking over the tether, but allow the second section 1040 to remain straight when an axial compressive force is applied to the positioning tool 1006. In some examples, the cutouts 1066 are arranged in a spiral pattern around the circumference of the positioning tool 1006. In some cases, the first section 1030 includes cutouts the same as, or similar to, the cutouts 1066 to allow the first section 1030 to flex/bend during tracking over tether while allowing the first section 1030 to remain straight when an axial compressive force is applied to the positioning tool 1006.

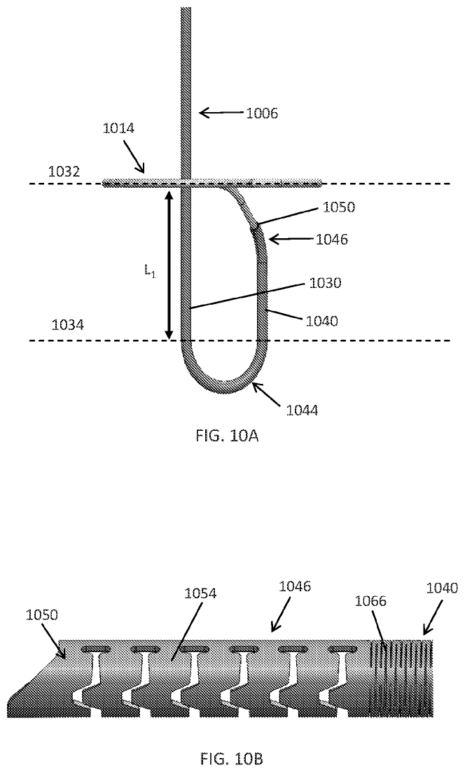

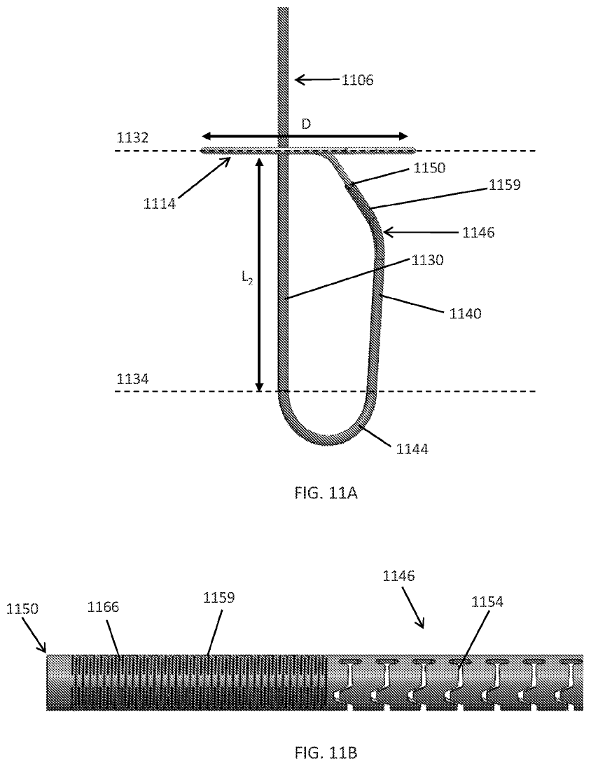

FIGS. 11A and 11B show another exemplary positioning tool 1106 having similar features as positioning tool 1006 but with a few different features. The length L₂ of the first section 1130 between the anchor plane 1132 and a plane 1134 where the curvature of the first preferentially bendable region 1144 begins is longer than the Li of the first section 1030 of the positioning tool 1006 in FIGS. 10A and 10B. This longer length may help to assure that the anchor 1114 is held high enough to provide room for prosthetic valve deployment. In some examples, the length L₂ ranges between any two of the following values: 25 mm, 30 mm, 35 mm, 40 mm, 45 mm, and 50 mm.

Compared to the positioning tool 1006 of FIGS. 10A and 10B, the second section 1140 can be non-parallel to the first section 1130. This angled arrangement can provide more room for deployment of the prosthetic valve (e.g., frame portion of the prosthetic valve). For example, this arrangement can position the distal end 1150 and the second preferentially bendable region 1146 farther away from the first section 1130, thereby reducing the chance of the distal end 1150 and/or the second preferentially bendable region 1146 from interacting with and causing misalignment of the valve prosthesis during deployment.

As shown in FIG. 11B, the distal end 1150 of the positioning tool 1106 can have a straight (e.g., non-beveled) edge. This straight configuration may prevent the distal edge from cutting into the tether. In addition, a distal section 1159 of the positioning tool 1106 can include a distal section 1159 that is distal to the second preferentially bendable region 1146. The distal section 1159 may provide more room for deployment of the prosthetic valve. The distal section 1159 can include cutouts 1166 that are configured to allow the distal section 1159 to flex/bend as it is tracked over the tether, but allow the distal section 1159 to remain straight when an axial compressive force is applied to the positioning tool 1106. In some examples, the cutouts 1166 are arranged in a spiral pattern around the circumference of the positioning tool 1106. In some cases, the first section 1130 and/or the second section 1140 include cutouts the same as, or similar to, the cutouts 1166 to allow the first section 1130 and/or the second section 1140 to flex/bend during tracking over tether while allowing the first section 1130 and/or the second section 1140 to remain straight when an axial compressive force is applied to the positioning tool 1106.

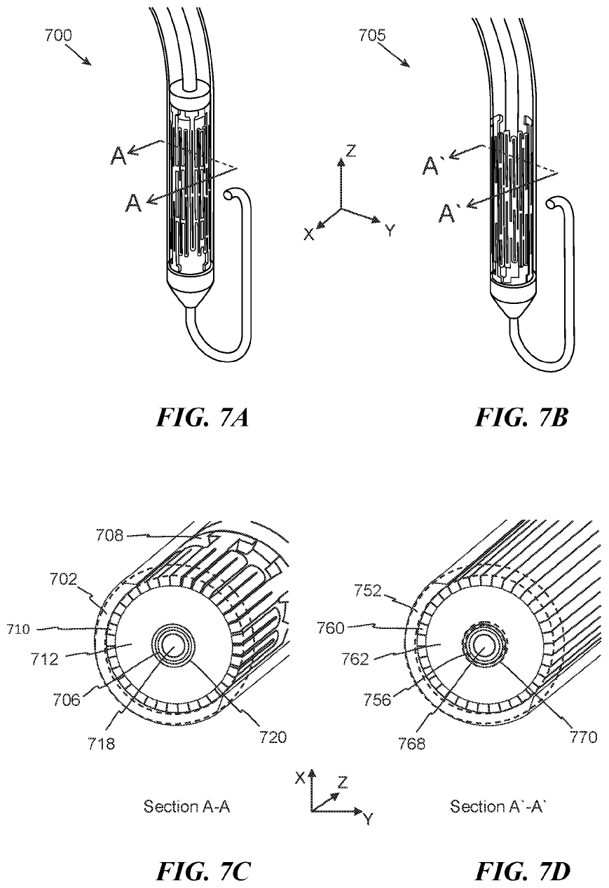

FIGS. 7A-7D show, in perspective and section views, examples of a prosthetic valve that is carried within a valve delivery catheter during delivery concentrically along a tether. In the exemplary concentric valve delivery catheter 700 (shown in FIG. 7A), the prosthetic valve is carried by a valve delivery member 708 at a distal portion of the valve delivery catheter 700. FIG. 7C illustrates an outer wall 702 (also referred to herein as the outer sheath) of the valve delivery catheter 700 and a valve delivery member 708 that positioned within the valve delivery catheter and retains the prosthetic valve about a central lumen 720. The central lumen is formed by an inner shaft of the valve delivery member that extends from a distal end (e.g., a nosecone portion) to a proximal end, and that is sized to accommodate a positioning tool 706 and a tether 718 for translating therein. The example prosthetic valve has a frame 710 and leaflet material 712 supported thereby and packed about the central lumen 720.

The exemplary concentric valve delivery catheter 705 of FIG. 7B is similar to the catheter 700 of FIG. 7A except that the valve delivery member is not present in this embodiment. In the example valve delivery catheter 705, the prosthetic valve is retained by a nosecone at a distal end of the valve delivery catheter. FIG. 7D illustrates an outer wall 752 (also referred to herein as the outer sheath) of the valve delivery catheter and an inner shaft 770 that defines a distal portion of a tether lumen. The tether lumen extends from a distal end of the valve delivery catheter to a proximal end (e.g., external to the patient), and is sized to accommodate a positioning tool 756 and a tether 768 for translating therein. The example prosthetic valve has a frame 760 and leaflet material 762 supported thereby and packaged about the central lumen. A nosecone is coupled to the distal end of the valve delivery catheter. In some embodiments, a valve delivery member includes the nosecone. The nosecone can include a port sized to accommodate the tether and the positioning tool. The port can be centrally located with respect to an outer perimeter (e.g., circumference) of the nosecone. When coupled, the port and the tether lumen can be located adjacent to and aligned with one another.

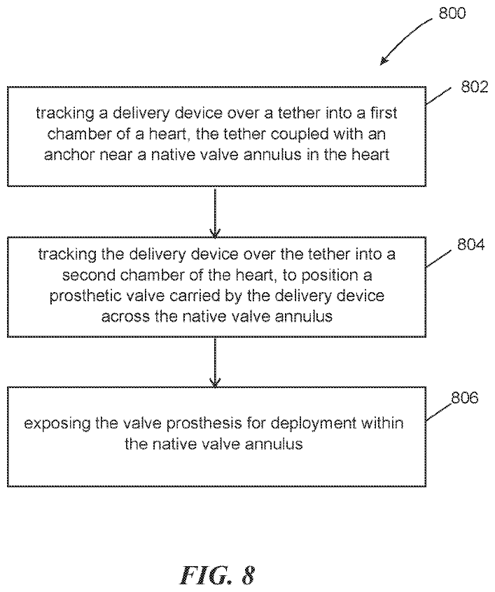

FIG. 8 is a flowchart 800 of a method of tracking a valve delivery catheter over a tether for deploying a prosthetic valve. An example operation 802 comprises tracking a delivery device over a tether into a first chamber of a heart, where the tether is coupled with an anchor that is near a native valve annulus in the heart. An example operation 804 comprises tracking the delivery device over the tether into a second chamber of the heart, to position a prosthetic valve capsule that is carried by the delivery device across the native valve annulus. An example operation 806 comprises exposing the valve prosthesis for deployment within the native valve annulus.

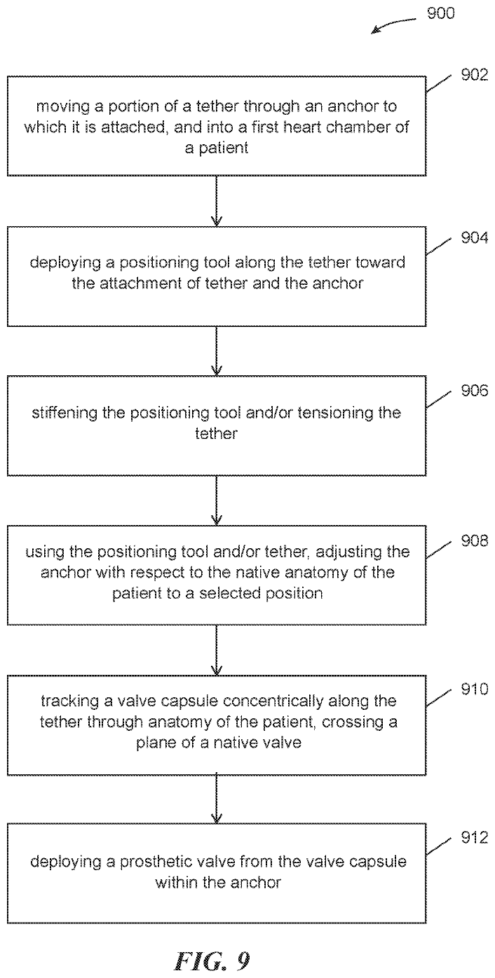

FIG. 9 is a flowchart 900 of a method of concentrically tracking a valve delivery catheter over a tether for deploying a prosthetic valve. An example operation 902 comprises moving a portion of a tether through an anchor to which it is attached, and into a chamber (e.g., ventricle) of a patient. An example operation 904 comprises deploying a positioning tool along the tether toward the anchor, or an attachment that is attached to the tether and/or a proximal end of the anchor. A distal edge of the positioning tool can then engage with a proximal edge of the anchor, or a proximal edge of the attachment that is attached to the tether and/or a proximal end of the anchor. An example operation 906 comprises stiffening the positioning tool and/or tensioning the tether. In some cases, the positioning tool takes on a curved shape when compressed/stiffened. For example, the positioning tool may take on a U-shaped bend sub-annular to the anchor so that a distal end of the positioning tool is directed toward the anchor. The distal end of the positioning tool can include an engagement surface that is configured to engage or couple directly or indirectly with a proximal end of the anchor. In this way, the stiffened positioning tool can engage with the anchor for controlling movement of the anchor. In some cases, the positioning tool additionally includes a second bend near the distal end of the positioning tool that bends radially inward to accommodate the geometry of the anchor.

An example operation 908 comprises using the positioning tool and/or tether, adjusting the position of the anchor with respect to the native anatomy of the patient to a selected position. For example, it may be desirable for the anchor to be as close to the annulus of the native valve as possible. In some cases, the anchor may be adjusted upward toward the annulus of the native valve. An example operation 910 comprises tracking a valve capsule concentrically along the tether through anatomy of the patient, crossing a plane of a native valve. An example operation 912 comprises deploying a prosthetic valve from the valve capsule within the anchor.

While the above are given as a sequence of operations, it will be appreciated that one or more of the operations can occur in a different sequence. For example, in some embodiments the stiffening of the positioning tool and/or tensioning of the tether is performed following tracking the valve delivery catheter across the native valve of the heart. For example, in some embodiments the deploying of the positioning tool is performed following tracking the valve delivery catheter across the native valve of the heart.

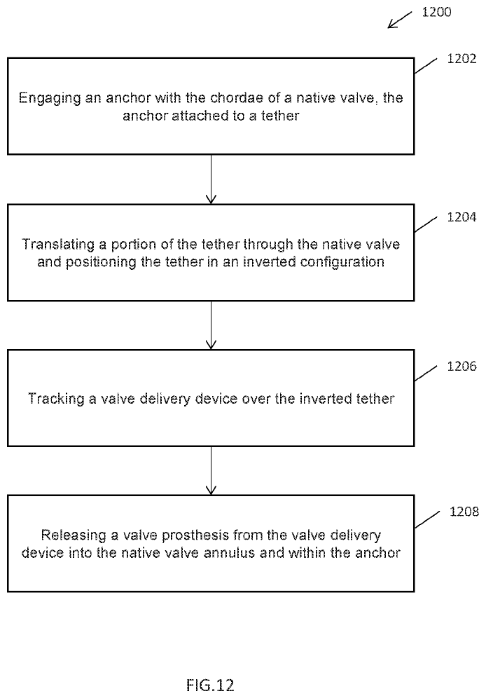

FIG. 12 is a flow chart 1200 indicating a method of delivering a valve prosthesis including inverting a tether for tracking the valve prosthesis. An example operation 1202 comprises engaging an anchor with the chordae tendineae of a native diseased valve. Deployment of the anchor may be accomplished using an anchor delivery device or system, which can include an anchor delivery catheter (also referred to as a steerable catheter or outer sheath) that can be configured to bend and straighten to steer the anchor into proper position. In some cases, the anchor delivery device or system may further include an anchor guide, which can translate within the steerable catheter. The anchor guide can include an inner lumen for housing the anchor therein. The anchor guide can take on a curved shape during deployment of the anchor from the anchor guide. In some examples, the anchor includes a wire that, when deployed, winds around a central axis and has a planar shape. The anchor may be deployed so that the windings encircle the chordae of the native valve. A proximal end of the anchor can be releasably attached to a tether that maintains connection to the anchor during later deployment of the valve prosthesis.

Once the anchor is deployed, at operation 1204, a portion of the tether can be translated through the native valve and positioned in an inverted configuration. In some examples, this is accomplished using the steerable catheter of the anchor delivery device or system. For example, while the steerable catheter is still tracked over the tether (e.g., after deployment of the anchor), the steerable catheter can be advanced through the valve annulus and the plane of the anchor. This advancement of the steerable catheter can cause a portion of the tether to translate from a first chamber of the heart (e.g., atrium) to a second chamber of the heart (e.g., ventricle). In some cases, positioning the tether in the inverted configuration comprises causing the tether to take on a U-shaped bend. In some cases, positioning the tether in the inverted configuration comprises translating the tether distally with respect to the steerable catheter to provide extra tether length within the second chamber.

Once the tether is in the inverted configuration, at operation 1206, a valve delivery device can be tracked over the tether. Tracking the valve delivery device over the tether can involve advancing the valve delivery device through the annulus of the native valve over the tether. The valve delivery device can include a positioning tool that can be used to adjust the axial height of the anchor relative to the native valve annulus (See, e.g., FIG. 9). The positioning tool may be configured to transition from a straight configuration (e.g., for traversing through the patient's vessels within the valve delivery device) to a pre-determined curved shape (e.g., U-shape). At operation 1208, the valve prosthesis can be released from the valve delivery device into the native valve annulus and within the anchor. Opposing forces between the prosthetic valve and the anchor can secure the prosthetic valve in place within the annulus of the native valve. Once the valve prosthesis is fully deployed, the valve delivery device and the tether can be removed from the heart and the patient's body.

The valve prosthesis can be similar to those of existing transcatheter-delivered valves. The valve prosthesis can be similar to existing surgical tissue valves, and mechanical valves. At least a portion of the valve segment may be positioned within at least a portion of the valve prosthesis, for example with a frame structure of the valve prosthesis. The valve segment may include leaflets formed of multi-layered materials for preferential function. The valve segment may comprise at least one leaflet having an inner layer and an outer layer. The valve segment may be attached directly to the valve prosthesis. Alternatively, the valve segment may be attached to an intermediate valve structure that is in turn connected to the valve prosthesis. The valve segment may be connected to the valve prosthesis before or after the valve prosthesis has been deployed adjacent a native valve. The valve prosthesis may be attached to a leaflet of the valve segment, for example an outer layer of a leaflet, at one or more ends of the valve prosthesis. The valve prosthesis may be attached to a leaflet of the valve segment, for example an outer layer of a leaflet, at one or more intermediate portions of the valve prosthesis. The valve segment may comprise a plurality of leaflets. The valve segment may comprise a biocompatible one-way valve. Flow in one direction may cause the leaflet(s) to deflect open and flow in the opposite direction may cause the leaflet(s) to close.

The frame structure may be configured like a stent. The frame structure may, for example, comprise a scaffold in a diamond pattern formed from a shape memory material (e.g., nitinol, NiTi). One of ordinary skill in the art will appreciate that many other structures, materials, and configurations may be employed for the frame structure. For example, the frame structure may be formed of a polymer of sufficient elasticity. The frame structure may be formed of a combination of metal and polymer, such as metal (e.g., shape memory material) covered in polymer. The frame structure may include a variety of patterns besides diamond shapes. In some embodiments, the frame structure is a closed frame such that blood flow is forced through valve segment therein. One or more skirts and/or seals may help force blood through the valve segment.

One of ordinary skill in the art will recognize based on the description herein that any of the valve prostheses described herein may comprise any of the frame structure shapes, frame structure designs, frame structure materials, anchor shapes, anchor windings, anchor materials, free end tips, leaflet(s) configurations, or any other of the variable features described herein in any combination thereof as desired.

### EXAMPLE 1

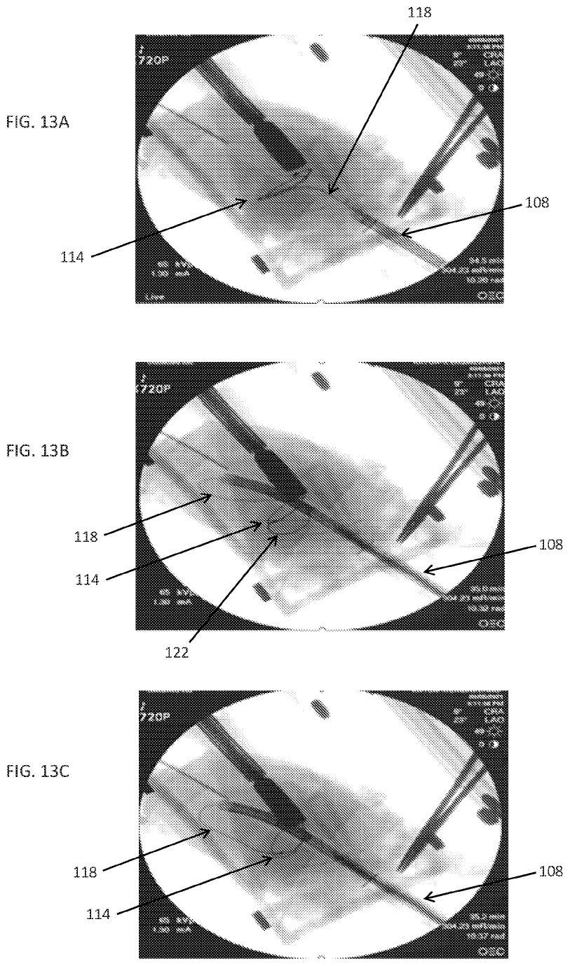

FIGS. 13A-13C show fluoroscopic images of an exemplary tether inversion procedure performed within a lamb heart. FIG. 13A shows the anchor 114 positioned around the chordae and attached to the tether 118 coming from the outer sheath 108 (e.g., like FIG. 2B). FIG. 13B shows the outer sheath 108 advanced toward the left ventricle apex. As shown, a slack 122 portion of the tether 118 is above the anchor 114 and the tether 118 can take on a constrained/twisted configuration due to tension (e.g., like FIG. 2F). FIG. 13C shows the tether 118 in an inverted configuration after partial retraction within the outer sheath 108 to remove the slack 122 and to allow the tether 118 to unwind into a U-shaped configuration sub-annular to the anchor 114 (e.g., like FIG. 2G).

### EXAMPLE 2

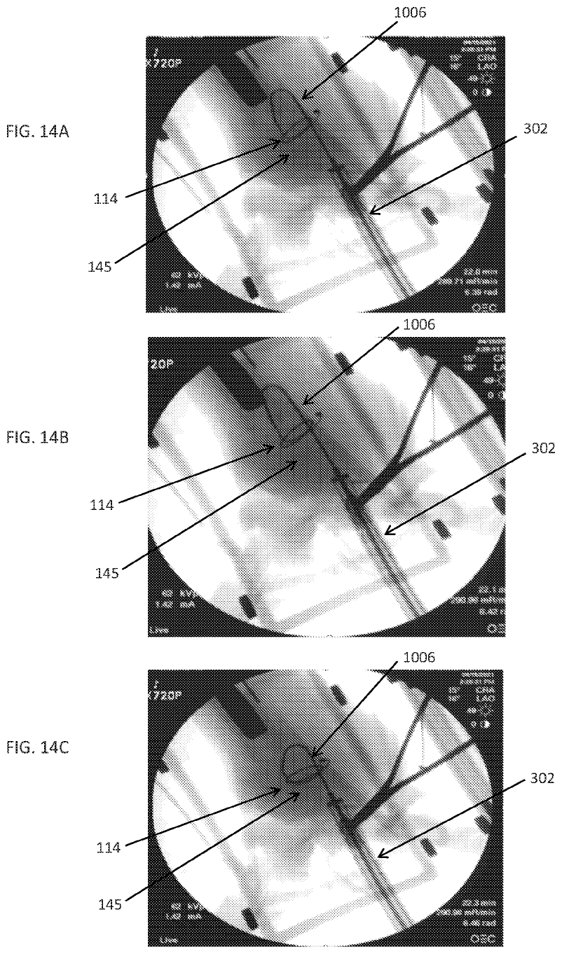

FIGS. 14A-14C show fluoroscopic images of an exemplary positioning tool 1006 used to control the axial height of an anchor 114 within a heart. These images show the anchor 114 positioned around the chordae and attached to a tether, wherein the positioning tool 1006 has been advanced from a valve delivery catheter 302 over the tether. The positioning tool 1006 is shown in a stiffened and curved state. FIG. 14A shows the positioning tool 1006 supporting the anchor 114 at first axial height relative to the native valve annulus 145. At FIG. 14B, the positioning tool 1006 has been pushed distally through the anchor 114, causing the anchor 114 to move distally to a second axial height that is farther from the native valve annulus 145. At FIG. 14C, the positioning tool 1006 has been pulled proximally while in the stiffened/bend configuration, causing the anchor 114 to move proximally to a third axial height that is closer to the native valve annulus 145. These images illustrate how the positioning tool 1006 can be used to control the axial height of the anchor 114.

### EXAMPLE 3

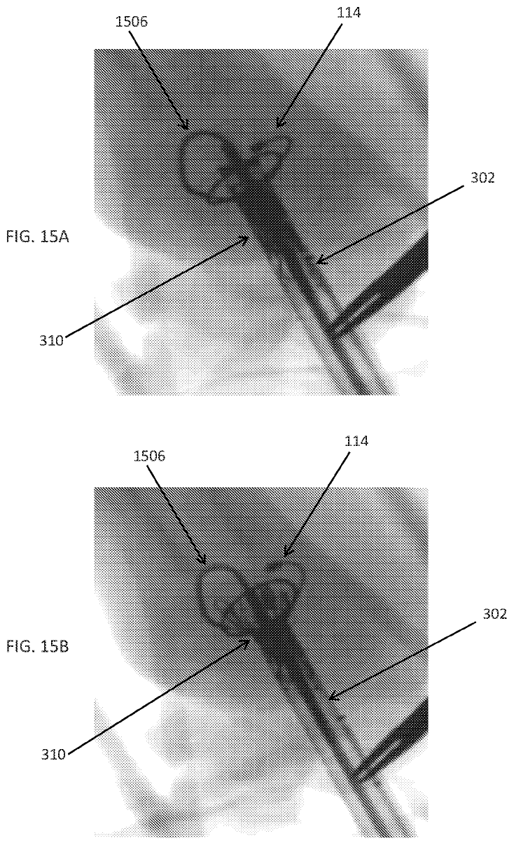

FIGS. 15A and 15B show fluoroscopic images of an exemplary deployment of a prosthetic valve 310 in a heart illustrating issues that may occur related to a length of the positioning tool 1506. As described above, the length (e.g., L₁ or L₂) of the positioning tool 1506 should be long enough so as not to interfere with deployment of the valve prosthesis 310. FIG. 15A shows the positioning tool 1506 supporting the anchor 114 close to the valve annulus prior to deployment of the valve prosthesis 310 from the valve delivery catheter 302. FIG. 15B shows the valve prosthesis 310 being advanced for deployment within the anchor 114. However, the U-shaped end of the positioning tool 1506 is too close to the anchor 114 to provide adequate room for deployment of the valve prosthesis 310. As described above, the length of the positioning tool 1506 can be chosen to be long enough to provide adequate room for valve prosthesis deployment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected,” “attached” or “coupled” to another feature or element, it can be directly connected, attached or coupled to the other feature or element or intervening features or elements may be present. In contrast, when a feature or element is referred to as being “directly connected,” “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “1”.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise,” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions.

For example, a numeric value may have a value that is +/−0.1% of the stated value (or range of values), +/−1% of the stated value (or range of values), +/−2% of the stated value (or range of values), +/−5% of the stated value (or range of values), or +/−10% of the stated value (or range of values). Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that these data represent endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure.

Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method for treating a diseased native valve in a patient, the method comprising:
encircling chordae of the diseased native valve with an anchor, the anchor having a tether attached thereto;
translating a portion of the tether through an annulus of the diseased native valve from a first chamber of the heart to a second chamber of the heart while the anchor is positioned around the chordae, wherein translating the tether causes the tether to form a bend within the second chamber;
tracking a valve delivery device over the tether; and
releasing a valve prosthesis from the valve delivery device into the annulus of the diseased native valve and within the anchor.

2. The method of claim 1, further comprising delivering the anchor to the diseased native valve with an anchor delivery device, the anchor delivery device comprising a steerable catheter.

3. The method of claim 2, wherein the translating the portion of the tether through the annulus of the diseased native valve comprises:
advancing the steerable catheter towards a plane of the anchor;
advancing the tether while maintaining its attachment to the anchor, generating slack in the tether that at least partially coils within the first chamber; and
advancing the steerable catheter across the plane of the anchor to position at least a portion of the tether into the second chamber.

4. The method of claim 3, wherein advancing the steerable catheter comprises advancing the steerable catheter to a position that is proximal to an apex of the second chamber.

5. The method of claim 3, further comprising retracting the anchor delivery device from the diseased native valve.

6. The method of claim 2, wherein the anchor delivery device further comprises an anchor guide that is configured to translate within the steerable catheter, wherein the anchor guide comprises an inner lumen for housing the anchor.

7. The method of claim 6, wherein the anchor guide takes on a curved shape during deployment of the anchor from the anchor guide.

8. The method of claim 1, wherein the tether takes on substantially a U-shaped bend within the second chamber when in a bent configuration.

9. The method of claim 8, wherein positioning the tether in the bent configuration comprises translating the tether distally with respect to a steerable catheter to provide increased tether length within the second chamber.

10. The method of claim 1, wherein tracking the valve delivery device over the tether comprises advancing a valve delivery catheter through the annulus of the diseased native valve.

11. The method of claim 1, wherein tracking the valve delivery device over the tether comprises deploying a positioning tool over the bent tether.

12. The method of claim 11, further comprising distally advancing the positioning tool until a distal end of the positioning tool interfaces with an attachment that is attached to a proximal end of the anchor.

13. The method of claim 11, further comprising applying a compression force along the positioning tool to cause the positioning tool to stiffen.

14. The method of claim 13, wherein applying the compression force comprises pulling the tether proximally to place tension on the tether.

15. The method of claim 14, wherein pulling the tether proximally to place tension on the tether comprises using a handle to apply a controlled degree of tension on the tether.

16. The method of claim 13, wherein applying the compression force causes the positioning tool to take on substantially a U-shaped bend sub-annular to the anchor.

17. The method of claim 16, further comprising releasably attaching a distal end of the positioning tool to the anchor.

18. The method of claim 11, further comprising adjusting a position of the anchor relative to the diseased native valve using the positioning tool.

19. The method of claim 18, wherein adjusting the position of the anchor comprises pulling the positioning tool proximally to move the anchor toward the annulus of the diseased valve.

20. The method of claim 18, wherein the position of the anchor is adjusted closer to the annulus of the diseased native valve.

21-127. (canceled)
